(19) United States
(12) Patent Application Publication
(10) Pub. No.: US 2012/0245079 A1
(43) Pub. Date: Sep. 27, 2012

(54) Treatment of Diseases Caused by Bacterial Exotoxins

(76) Inventors: David M. Kranz, Champaign, IL (US); Patrick Schlievert, Iowa City, IA (US)

(21) Appl. No.: 13/393,603

(22) PCT Filed: Sep. 3, 2010
(86) PCT No.: PCT/US10/47782

§ 371 Date: Jun. 5, 2012

Related U.S. Application Data
(60) Provisional application No. 61/239,638, filed on Sep. 3, 2009.

(51) Int. Cl. A61K 38/17; A61P 17/02; C07K 14/47; A61P 17/00; A61P 31/04; A61P 29/00; A61K 38/02; A61P 11/00

(52) U.S. Cl. 514/1.6; 514/21.2; 514/1.1; 514/18.6; 514/2.7; 530/350

(57) ABSTRACT

Provided are high affinity T cell receptor variable regions that are useful for treating diseases caused by superantigens including atopic dermatitis, pneumonia and delayed wound healing. The variable regions contain mutants that result in high affinity binding to the superantigen.

### CROSS-REFERENCE TO RELATED APPLICATIONS

This application claims priority to U.S. provisional patent application Ser. No. 61/239,638, filed Sep. 3, 2009 which is hereby incorporated by reference in its entirety to the extent not inconsistent with the disclosure herewith.

### STATEMENT REGARDING FEDERAL FUNDING

This invention was made with U.S. Government support under Grant number R01 AI064611 awarded by the National Institutes of Health. The U.S. Government has certain rights in the invention.

### STATEMENT REGARDING SEQUENCE LISTING

The sequence listing included herewith in text format is considered a part of the application and disclosure as filed.

### BACKGROUND OF THE INVENTION

Toxic shock syndrome (TSS) was characterized as a disease associated with staphylococci infection over 25 years ago. Subsequently, toxic shock syndrome toxin-1 (TSST-1) from Staphylococcus aureus was identified as the protein responsible for the disease in most cases. TSST-1 is a member of a family of molecules secreted by S. aureus and Streptococcus pyogenes that cause elevated systemic cytokine levels, including tumor necrosis factor-α (TNF-α) and interleukin-1 (IL-1), leading to fever, TSS, and ultimately organ failure. The term superantigen (SAg) was given to this class of molecules because these toxins stimulate a large fraction of T cells bearing the same variable regions of the T cell receptor beta chain (Vβ regions). As up to 20% of the T cell repertoire can bear the same Vβ region, SAgs are capable of stimulating thousands of times more T cells than conventional antigens. Since soluble monovalent ligands for the T cell receptor (TCR) cannot themselves stimulate T cells, SAgs act by cell-to-cell cross-linking TCRs and class II major histocompatibility complex (MHC) molecules on antigen presenting cells.

The bacterial SAg family now contains over 20 members, including the S. aureus enterotoxins TSST-1, (SE) A to E, and G to Q and the Streptococcus pyogenes exotoxins A (Spe) A, C, G to M, streptococcal superantigen (SSA) and the mitogenic exotoxins called SMEZ. Sequence based phylogenetic relationships among these toxins indicated that they represent five groups, in which one group contains TSST-1 as the only known member. The structures of SAgs, including TSST-1, have been shown to be very similar. A smaller N-terminal domain contains two β-sheets and a larger C-terminal domain consists of a central α-helix and a five-stranded β-sheet. Although all bacterial SAgs share a common three-dimensional structure, they exhibit diversity in their specificities for TCR Vβ domains and class II MHC molecules, as well as in the molecular architecture of the respective MHC-SAg-TCR signaling complexes that they form.

These superantigens cause and contribute to many diseases, including pneumonia, mastitis, phlebitis, meningitis, urinary tract infections, osteomyelitis, endocarditis, nosocomial infection, staphylococcal food poisoning and toxic shock syndrome. Current treatments include supportive care, antibiotics, and intravenous immune globulin. There are several strains of S. aureus that are highly antibiotic resistant.

Staphylococcal enterotoxin B (SEB), one of the more thoroughly characterized SAgs, has been considered a potential biological weapon due to its toxicity and to previous programs involving large-scale production and aerosolization.

Despite the fact that the molecular interactions of these toxins have been well-characterized, therapeutics capable of neutralizing their activity are not available for clinical use.

As described above, secreted bacterial toxins (exotoxins) from Staphylococcus aureus and Streptococcus pyogenes cause serious clinical problems including toxic shock syndrome. In particular, one of the toxins called toxic shock syndrome toxin-1 (TSST-1) is the cause of deaths due to menstrual-associated and one-half of non-menstrual infections. Toxic shock syndrome is characterized by many symptoms (fever, hypotension, rash etc), ultimately leading to major organ failure and death. It is now an especially dangerous disease due to the presence of antibiotic resistant strains of S. aureus (Methicillin Resistant S. aureus, MRSA). These toxins act as “Superantigens” by binding simultaneously to a receptor on T cells (the T cell receptor Variable beta region) and on another immune cell (a protein encode by the major histocompatibility complex), thereby triggering the massive release of cytokines. These cytokines act in many different ways, leading to toxic shock syndrome. It is now clear that various other serious diseases are also caused, or made worse, by the presence of the bacterial toxins. This includes pneumonia caused by pulmonary infections, severe atopic dermatitis, and wound healing. There is a need for treatments of these diseases and neutralization of the toxins.

### SUMMARY OF THE INVENTION

This disclosure is generally directed to the use of V beta drugs for treating or improving conditions caused or exacerbated by bacterial superantigens, including improving healing, neutralizing bacterial toxins, and controlling related inflammation, among other uses. In an embodiment, the invention provides therapeutic agents for use in the in vivo treatment of diseases or disorders. The terms “high-affinity neutralizing agent”, “V beta drug” and “Vβ drug” and other forms of these terms are used interchangeably herein.

More specifically, provided in an embodiment are drugs for the treatment of humans or other mammals that are exposed to one or more superantigens. In an embodiment, provided are drugs for the treatment of humans or other mammals that are exposed to one or more toxins SEB, SEC, SpeA and/or TSST-1, and in an embodiment, the use of these drugs in the treatment of the diseases described herein. The drugs (referred to as V beta drugs) are small soluble protein domains (about 12,000 daltons in an embodiment) of the T cell receptor Variable beta region that have been engineered to have very high binding affinity for SEB, SEC, SpeA or TSST-1 (picomolar binding constants, for example). The drugs are believed to bind to the active site of the toxins and thereby prevent the toxins' normal effects in vivo.

The V beta drugs described herein are useful for treatment of disorders or diseases which are caused by or at least partially contributed to by superantigens. In an embodiment, the V beta drugs described herein are useful in treating delayed wound healing, pneumonia, CA-MRSA, pulmonary TSS, necrotizing pneumonia, purpura fulminans, extreme pyrexia, or atopic dermatitis, among other uses.

Provided in an embodiment are high affinity mutants of a T cell receptor variable beta region (“V beta drugs”) that have very high affinity for toxins described herein. In separate embodiments, the V beta drugs of the invention have equilibrium binding constant K_{D} for the bacterial superantigen of between about 10⁻⁸M and 10⁻¹²M. As used herein, the word “about” when referring to a numerical value also includes the numerical value itself. For example, “between about 10⁻⁸M and 10⁻¹²M” includes the value 10⁻⁸M for any purpose, including the insertion or deletion of the value in a claim. In other embodiments, the V beta drugs of the invention have equilibrium binding constant K_{D} for the bacterial superantigen of less than 10⁻¹² molar. These binding constants lead to effective neutralization at the very doses of toxins that are incapacitating or lethal. One advantage to the invention is that the compositions described herein can be used to treat incapacitating or lethal exposure to the toxins in a range of diseases.

In an embodiment, provided is a high-affinity neutralizing agent for the toxin SEB and SEC for use in treating pulmonary pneumonia, atopic dermatitis and skin hypersensitivity reactions, and wound healing that is delayed by the presence of the toxin. Also provided is the use of a high-affinity neutralizing agent for the toxin TSST-1 for use in treating pneumonia and atopic dermatitis. Also provided is the use of a high-affinity neutralizing agent for the toxin TSST-1 for use in treating a skin hypersensitivity reaction and improve wound healing. Also provided is the use of a high-affinity neutralizing agent for the toxin SpeA for use in treating a disease or disorder resulting from this toxin, including streptococcal toxic shock syndrome with or without necrotizing fasciitis, sepsis without toxic shock syndrome, scarlet fever, erysipelas, rheumatic fever, acute glomerulonephritis, certain forms of psoriasis, and various neuropsychiatric disorders (PANDAS; for example obsessive compulsive disorder, Tourette's Syndrome, and Sydenham's chorea).

It is known in the art that the superantigens described herein cause and contribute to a number of diseases or disorders. The Vbeta drugs described herein and functional equivalents are used to treat one or more of the diseases or disorders resulting from the superantigens that may be exhibited or detectable or present in a patient. The disclosure herein is intended to include all such diseases or disorders, even if the disease or disorder is not specifically listed. As used herein, a sequence or drug that is a “functional equivalent” of another sequence or drug has the same or similar function in a given system. Some examples of functional equivalents of a Vbeta drug disclosed herein include proteins with mutations that do not affect the function of the drug to neutralize a superantigen. The presence of and identity of these mutations are easily understood and determined by one of ordinary skill in the art. Other examples of functional equivalents include proteins that share 90% sequence identity with the V beta drugs disclosed. Other examples of functional equivalents include proteins that share 95% sequence identity with the V beta drugs disclosed. Other examples of functional equivalents include proteins that share 97% sequence identity with the V beta drugs disclosed. Functional equivalents of the V beta drugs disclosed herein are intended to be included. As used herein “neutralize” is used to indicate a reduction in effect, not necessarily a complete inactivation.

More specifically, provided is a method of treating a disease caused by a bacterial exotoxin in a mammal comprising: administering an effective amount of a high affinity mutant of a T cell receptor variable region to a mammal, wherein the high affinity mutant of a T cell receptor variable region binds to a bacterial superantigen with high affinity. In an further embodiment, provided is a method of treating a disease caused by a bacterial superantigen in a mammal comprising: administering an effective amount of a Vβ drug to a mammal, wherein the Vβ drug has an equilibrium binding constant K_{D} for the bacterial superantigen of between about 10⁻⁸M and 10⁻¹²M. In an embodiment, the disease is one or more of delayed wound healing, pneumonia including necrotizing pneumonia, community associated (“CA”)-MRSA, pulmonary TSS, necrotizing fasciitis, purpura fulminans, extreme pyrexia, or atopic dermatitis and forms of psoriasis. It has been shown that the high affinity mutant of a T cell receptor variable region binds to superantigens on a one:one molar ratio to neutralize their toxicity, in an embodiment. This information is useful for determining dosage ranges, for example.

In an embodiment, the high affinity mutant of a T cell receptor variable region described herein is a V beta drug. In an embodiment, the V beta drug is G5-8. In an embodiment, the V beta drug comprises the sequence of SEQ ID No.: 76. In an embodiment, the V beta drug is D10. In an embodiment, the V beta drug comprises the sequence of SEQ ID No.: 74. In an embodiment, the V beta drug is L3. In an embodiment, the V beta drug comprises the sequence of SEQ ID No.: 77. In an embodiment, the V beta drug is D10V. In an embodiment, the V beta drug comprises the sequence of SEQ ID No.: 75. In an embodiment, the high affinity mutant is isolated or purified.

In an aspect of the invention, the V beta drug is used in combination with a conventional treatment for the disease or disorder such as an antibiotic; supportive care, for example, administration of fluid and/or electrolytes to maintain blood pressure; and use of pharmaceutically active compounds such as vasopressors to maintain blood pressure, activated protein C to slow clotting, or intravenous immunoglobulin (IVIG), for example. In an embodiment, the high affinity mutant of a T cell receptor exhibits an equilibrium binding constant K_{D} for a bacterial superantigen of between about 10⁻⁸M and 10⁻¹²M. In an embodiment, the mammal treated is a human. In an embodiment, the mammal treated is a dog. In an embodiment, the mammal treated is another mammal. Also provided is a V beta drug for use in therapy. In an embodiment, the V beta drug is used for the treatment of one or more of delayed wound healing, pneumonia, CA-MRSA, pulmonary TSS, necrotizing pneumonia, purpura fulminans, extreme pyrexia, or atopic dermatitis. In an embodiment, the V beta drug is D10, D10V, G5-8, or L3, or combinations thereof.

Also provided is a kit for treatment of a disorder caused by a bacterial superantigen in a sample, comprising a compound comprising SEQ ID. NO: 74-77 or a functional equivalent thereof. In an embodiment of this aspect of the invention, the sample is a mammal. In an embodiment of this aspect of the invention, the sample is a human. In an embodiment of this aspect of the invention, the sample is a blood, tissue or other sample taken from a mammal such as a human. In an embodiment of this aspect of the invention the compound is D10, L3, D10V or G5-8 or combinations thereof.

Also provided is a method of treating bacterial toxicity caused by one or more SAgs comprising the step of administering an effective amount of a V beta drug to an individual diagnosed with or suspected of having toxicity caused by one or more bacterial SAgs, wherein the V beta drug is capable of binding to a bacterial SAg. In an embodiment, the toxicity is caused by Staphylococcus aureus or Streptococcus pyogenes. In an embodiment, the toxicity is manifested by a diagnosis or suspected diagnosis of one or more of delayed wound healing, pneumonia, CA-MRSA, pulmonary TSS, necrotizing pneumonia, purpura fulminans, extreme pyrexia, or atopic dermatitis. In an aspect of the invention, the bacterial exotoxin is secreted by MRSA. In an aspect of the invention, the bacterial exotoxin is secreted by MSSA. In an embodiment, the Vβ drug is able to bind to staphylococcal enterotoxin B. In an embodiment, the Vβ drug is able to bind to TSS toxin-1. In an embodiment, the Vβ drug is able to bind to staphylococcal enterotoxin C. In an embodiment, the Vβ drug has a binding affinity to a bacterial SAg equal to or greater than natural (wild-type) T cell receptors. In an embodiment, the Vβ drug contains the amino acid sequence of the G5-8 Vbeta protein. In an embodiment, the Vβ drug contains the amino acid sequence of the D10 Vbeta protein. In an embodiment, the Vβ drug contains the amino acid sequence of the D10V Vbeta protein. In an embodiment, the Vβ drug contains the amino acid sequence of the L3 Vbeta protein. In an embodiment, the toxicity is caused by multiple bacterial SAgs.

In an embodiment, the Vβ drugs D10V or L3 are used in the methods of the invention. In an embodiment, V beta drugs G5-8 or D10 are used in the methods of the invention. G5-8 (sometimes also referred to as S4-8), a mouse Vβ domain (Vβ38.2) engineered to neutralize SEB with 48 pM affinity was generated as described in Buonpane et al. “Neutralization of staphylococcal enterotoxin B by soluble, high-affinity receptor antagonists,” Nat Med 2007; 13:725-9. D10, a human Vβ domain (Vβ32.1) engineered to neutralize TSST-1 with 180 pM affinity was generated as described in Buonpane et al. “Characterization of T cell receptors engineered for high affinity against toxic shock syndrome toxin-1,” J Mol Biol 2005; 353:308-21. A modification of these methods can be used to generate the other Vβ drugs described herein without undue experimentation. In an embodiment, the high affinity V beta drug of the invention is prepared using the method described in U.S. Pat. No. 6,759,243, Buonpane (2005), J. Mol. Biol. 353(3): 308-321, or PCT/US 07/64085. Methods of making the TCR V beta regions generally comprise: (a) cloning the T cell receptor variable region gene in a yeast display vector; (b) mutagenizing the T cell receptor variable region to generate a library of mutants; and (c) selecting the mutants which have the highest binding affinity to a ligand. Steps (b) and (c) can be repeated as desired, in order to obtain a T cell receptor variable region having the desired stability. Further aspects are described elsewhere herein. Other methods for generating G5-8, L3, D10V, D10 and other Vβ drugs can be used, as will be appreciated by one of ordinary skill in the art.

In an embodiment, provided is a compound or composition for use as a medicament. In an embodiment, provided is a compound or composition for its use in therapy. In separate embodiments, provided is a mutant TCR V beta region for use in treating atopic dermatitis, improving wound healing, or treating pneumonia and other illnesses listed above. In an embodiment, the compound or composition is isolated or purified.

As used herein, a high affinity V beta drug has higher affinity for a toxin, in an embodiment SEB or TSST-1, greater than the wild type V beta region.

Also provided is the use of a soluble mutant T cell receptor (TCR) variable region having higher affinity than the wild type T cell receptor variable region for a bacterial superantigen to treat disease, wherein said T cell receptor variable region is a mutant T cell receptor variable region carrying one or more mutations in a TCR variable region. In one embodiment, the TCR variable region exhibits an equilibrium binding constant K_{D} for the bacterial superantigen of between about 10⁻⁸M and 10⁻¹²M. In one embodiment, the TCR variable region is a mutant TCR having one or more mutations in a CDR. In one embodiment, the TCR variable region is a mutant TCR having one or more mutations in a FR region. In one embodiment, the bacterial superantigen is toxic shock syndrome toxin-1. In one embodiment, the TCR variable region has one or more mutations in the human Vβ2 region. In one embodiment, the TCR variable region has one or more mutations in the Vβ2.1 region. In one embodiment, the TCR variable region has one or more mutations in CDR2. In one embodiment, the bacterial superantigen is staphylococcal enterotoxin B. In one embodiment, the TCR variable region has one or more mutations in the mouse Vβ8 domain. In one embodiment, the TCR variable region has one or more mutations in the Vβ8.2 domain.

Superantigen toxicity is manifested by a diagnosis or suspected diagnosis of disorders such as pneumonia, purpura fulminans, necrotizing pneumonia, pneumonia TSS, extreme pyrexia, atopic dermatitis, or delayed wound healing. The association of the diseases with superantigens has not been clear or obvious. These illnesses have only been recently described in association with superantigens. In some cases, such as purpura fulminans and extreme pyrexia, the association of the illnesses with Staphylococcus aureus infection has not been described until recently (see Kravitz, G. R., D. J. Dreis, M. L. Peterson, and P. M. Schlievert. 2005. Purpura fulminans due to Staphylococcus aureus. Clin. Infect. Dis. 40:941-947; Assimacopoulos, A. P., K. L. Strandberg, J. H. Rotschafer, and P. M. Schlievert. 2009. Extreme pyrexia and rapid death due to Staphylococcus aureus infection: analysis of two cases. Clin. Infect. Dis. 48:612-4). In certain diseases, for example, atopic dermatitis and delayed wound healing, the use of the neutralizing agents described herein was used to help show these toxins are involved in the diseases. Thus, the extent to which the toxins caused these diseases, or even pneumonia, was unclear until these studies, and thus could not have been anticipated or predicted.

These illnesses have also been demonstrated recently in the rabbit model of human disease (see materials provided elsewhere herein and John, C. C., M. Niermann, B. Sharon, M. L. Peterson, D. M. Kranz, and P. M. Schlievert, 2009. Staphylococcal toxic shock syndrome erythroderma is associated with superantigenicity and hypersensitivity. Clin. Infect. Dis. 49:1893-6; Assimacopoulos et al (see paper cited above for discussion of extreme pyrexia)). It is generally regarded that rabbits are a highly sensitive excellent model for studies of the biological toxicity of superantigens (Schlievert, P. M. 2009. Cytolysins, superantigens, and pneumonia due to community-associated methicillin-resistant Staphylococcus aureus. J. Infect. Dis. 200:676-8; Lee, P. K., J. R. Deringer, B. N. Kreiswirth, R. P. Novick, and P. M. Schlievert. 1991). Fluid replacement and polymyxin B protection of rabbits challenged subcutaneously with toxic shock syndrome toxins. Infect. Immun. 59:879-884). Rabbits, unlike rodents and non-human primates, are approximately equivalently susceptible to superantigens as are humans and therefore a rabbit model of these diseases is considered representative of a human.

### BRIEF DESCRIPTION OF THE FIGURES

Those of skill in the art will understand that the figures, described below, are for illustrative purposes only. The figures are not intended to limit the scope of the present teachings in any way.

FIG. 1 shows the skin test reactivity of a rabbit to SEB, and neutralization by soluble high-affinity Vβ-TCR (G5-8). Rabbits were immunized subcutaneously in the nape of the neck for two weeks with SEB (25 ug) emulsified in incomplete adjuvant and then challenged intradermally with 0.1 ugSEB/0.1 ml volume with (A) 10 ug Vβ-TCR or (B) without Vβ-TCR. Skin test reactions were photographed after 24 hr.

FIG. 2 shows Immunization against SAg or administration of soluble high affinity Vβ-TCR G5-8 protects rabbits from lethal CA-MRSA pulmonary TSS. A, non-immunized and TSST-1-immunized rabbits alive after challenge with TSST-1+ USA200 strain; B, non-immunized and SEC-hyperimmunized rabbits alive after challenge with SEC+ USA400 strain; and C, PBS− and high affinity Vβ-TCR (G5-8)-treated rabbits alive after challenge with SEB+ USA400 strain.

FIG. 3 shows Rabbit lungs after exposure to CA-MRSA USA200 (TSST-1+, α-toxin−, β-toxin−, γ-toxin−, and PVL−) A-B, Left and right lungs removed from a non-immunized rabbit that received CA-MRSA USA200; C-D, Left and right lungs from a rabbit hyperimmunized against purified TSST-1 prior to administration of CA-MRSA USA200; and E-F, Left and right lungs removed from a rabbit exposed to PBS.

FIG. 4 shows Rabbit lungs after exposure to CA-MRSA USA400 MW2 (SEC+, α-toxin+, β-toxin−, γ-toxin+, and PVL+) or purified SEC. A-B, Left and right lungs from a non-immune rabbit that received MW2; C-D, Left and right lungs from a rabbit hyperimmunized against purified SEC prior to administration of MW2; E-F, Left and right lungs from a non-immune rabbit after intra-bronchial administration of 200 μg purified SEC; G-H, Left and right lungs from a rabbit hyperimmunized against purified SEC prior to administration of 200 μg purified SEC; and I-J, Left and right lungs from rabbit administered PBS.

FIG. 5 shows Histology of lung sections from rabbits challenged with CA-MRSA USA200 MNPA (TSST-1+, a-toxin−, β-toxin−, γ-toxin−, and PVL−). A-B, H&E staining of sections from a rabbit (lung image shown in FIG. 2A-B) that received CA-MRSA; C-D, H&E staining of sections from a rabbit (lung image shown in FIG. 2C-D) hyperimmunized against purified TSST-1 prior to administration of CA-MRSA; and E-F, H&E staining of sections from a rabbit (lung image shown in FIG. 2E-F) challenged with PBS.

FIG. 6 shows Histology of lung sections from rabbits challenged with CA-MRSA MW2 (SEC+, a-toxin+, β-toxin−, γ-toxin+, and PVL+) or purified SEC. A-B, H&E staining of sections from a non-immune rabbit (lung images shown in FIG. 3A-B) that received MW2; C-D, H&E staining of sections from a rabbit (lung images shown in FIG. 3C-D) hyperimmunized against purified SEC prior to administration of MW2; E-F, H&E staining of sections from a rabbit (lung images shown in FIG. 3E-F) that received 200 μg purified SEC; G-H, H&E staining of sections from a rabbit (lung images shown in FIG. 3G-H) hyperimmunized against purified SEC prior to administration of 200 μg purified SEC; and I-J, H&E staining of sections from a rabbit (lung images shown in FIG. 3I-J) administered PBS.

FIG. 7 shows soluble high affinity Vβ-TCR (G5-8) protects rabbits from respiratory distress and lethal TSS due to intra-bronchial purified SEB (100 or 200 μg, or 5 μg plus endotoxin enhancement). A, PBS or high affinity Vβ-TCR (G5-8)-treated rabbits alive after intra-bronchial administration of 100 (3 rabbits) or 200 μg SEB (3 rabbits) Note: all rabbits that received Vβ-TCR G5-8 were administered 200 μg purified SEB; B, PBS or high affinity Vβ-TCR (G5-8)-treated rabbits alive after intra-bronchial administration of 5 μg/kg purified SEB and 0.5 μg/kg endotoxin enhancement. Note: in the endotoxin enhancement experiments, high affinity Vβ-TCR G5-8 was administered iv at same time as intra-bronchial SEB (3 rabbits each received 50 μg G5-8), or 2 hrs after SEB administration (3 rabbits each received 100 μg G5-8).

FIG. 8 shows prevention of skin hypersensitivity reactions caused by TSST-1 with Vβ drug D10.

FIG. 9 shows wound healing with G5-8 when SEB is present.

FIG. 10 shows sequences of some hVβ2.1 mutants isolated in the yeast display system. The designation EP refers to clones isolated from the error-prone (stability) library. The designation R refers to clones isolated from the CDR2 (affinity) library. The designation C or D refer to clones isolated from the third and fourth sorts, respectively, from the combined CDR1, CDR2b, or HV4 (off-rate) library.

FIG. 11 shows sequences of Vβ8 mutants at the different stages of affinity maturation. G1 through G5 refers to the generation of clone isolated by yeast display. mTCR15 refers to a single-site mutant that has improved display on yeast, compared to the wild type Vβ8.2. CDR1, CDR2, HV4, and CDR3 regions are highlighted from left to right. Clones that were isolated multiple times are indicated with an asterisk.

FIG. 12 shows the sequences of some mVβ8.2 mutants isolated for binding to SEB.

FIG. 13 shows In Vitro inhibition of SEC and TSST-1 by High-Affinity Vβ Proteins L3 and D10V. A. Activity of soluble high-affinity Vβ mutants against SEC3. mTCR15 is the low affinity, wild type Vbeta. The drug L3 has been tested in vivo. B. Activity of soluble high-affinity Vb mutants against TSST-1. The drug D10V has been tested in vivo.

FIG. 14 shows High-Affinity Vβ drug L3 Neutralizes both SEC and SEB. A. Activity of soluble high-affinity Vβ mutants L3 and G5-8 against SEC3. B. Activity of soluble high-affinity Vβ mutants L3 and G5-8 against SEB. L3 neutralizes both SEC and SEB, while G5-8 neutralizes only SEB.

FIG. 15 shows the ability of D10V (left hand side of the figure) and L3 (right hand side of the figure) to neutralize TSST-1 as administered to rabbits. The figure shows the effect of the Vβ drugs (“VB” or “VB-TCR”) vs. the control PBS on the fever that results from exposure of TSST-1 and SEC and the survival of the rabbits. Rabbits were administered 5 μg/kg purified SEC3 or TSST-1 intravenously and then fever recorded over a 4 hour period (change in temperature reported) and 0.5 μg/kg endotoxin enhancement. Note: in the endotoxin enhancement experiments, high affinity Vβ-TCR was administered iv at same time as intra-bronchial superantigen (9 rabbits each received D10V or L3). Endotoxin was administered at the 4 hour time point (see procedures in Schlievert, P. M. 1982. Enhancement of host susceptibility to lethal endotoxin shock by staphylococcal pyrogenic exotoxin type C. Infect. Immun. 36:123-128).

FIG. 16 shows the ability of L3 to prevent lethality of community associated MRSA (SEC+) and SEC as administered to rabbits. The figure shows Day 2 fevers in rabbits treated with SEC in subcutaneous miniosmotic pumps or SEC plus VBetaTCR L3 given IV daily.

FIG. 17 shows Day 1 fevers in rabbits treated intrapulmonary with USA400 S. aureus (SEC+) and USA400 S. aureus plus L3 IV (500 ug/day)

### DETAILED DESCRIPTION OF THE INVENTION

Various features discussed herein in relation to one or more of the exemplary embodiments may be incorporated into any of the described aspects of the present invention alone or in any combination. Certain exemplary aspects of the invention are set forth herein. It should be understood that these aspects are presented merely to provide the reader with a brief summary of certain forms the invention might take and that these aspects are not intended to limit the scope of the invention. Indeed, the invention may encompass a variety of aspects that may not be explicitly set forth herein as would be understood by one of ordinary skill in the relevant art without undue experimentation.

The definitions and methods are provided to better define the invention and to guide those of ordinary skill in the art in the practice of the invention. Unless otherwise noted, the terms and phrases used herein have their art-recognized meaning, which can be found by reference to standard texts, journal references and contexts known to those skilled in the relevant art.

In one embodiment, the T cell receptor variable region is a human Vβ. The ligand can be any desired ligand, including an antigen or superantigen. In one embodiment, the ligand is an antibody for the T cell receptor variable region. In one embodiment, the ligand is a superantigen. In one embodiment, the ligand is TSST-1. In one embodiment, the ligand is SEB. In a specific embodiment, the T cell receptor variable region is hVβ2. In a specific embodiment, the T cell receptor variable region is mVβ8. Also provided is a stabilized T cell receptor variable domain comprising: a T cell receptor variable region which contains one or more mutations wherein the stabilized T cell receptor variable domain binds with greater affinity to a ligand than wild type. In a specific embodiment, the variable domain is hVβ. In a specific embodiment, the variable domain contains at least one mutation selected from the group consisting of: S88G, R10M, A13V, L72P, and R113Q. In a specific embodiment, the variable domain is mVβ8, and the variable domain contains the mutation G17E and optionally one or more mutations selected from the group consisting of: N24K, G42E, H47F, Y48M, Y50H, A52I, G53R, S54N, and T55V. In a specific embodiment, the variable domain is Vb2 and contains at least one mutation selected from the group consisting of: S52aF, K53N or E61V. In a specific embodiment, the variable domain is Vb2 and contains at least one mutation selected from the group consisting of: Q51E or V61T. In a specific embodiment, the variable domain is Vb8 and contains at least one mutation selected from the group consisting of: H28Y, A52I, G53R, G53K, or S54N. In a specific embodiment, the variable domain is Vb8 and contains at least one mutation selected from the group consisting of: A52V or S54N. Any mutation described here is intended to be disclosed to the extent it is described separately, for example if needed to remove or add a mutation in a claim. Any mutation or combination of mutations described or shown that gives a stabilized T cell receptor variable region is intended to be disclosed separately. Any mutation or combination of mutations described or shown that gives a high affinity mutant is disclosed separately.

Also provided is a method for using stabilized T cell receptor variable region to select mutants that bind to a ligand or molecule of interest with higher affinity than wild type comprising: providing a stabilized T cell receptor variable region; mutating the stabilized T cell receptor variable region to create a variegated population of mutants; contacting the variegated population of mutants with a ligand; and selecting those mutants which bind to the ligand with higher affinity than wild type. In one embodiment, the high affinity mutant and ligand bind with an equilibrium binding constant K_{D}<1 μM. In one embodiment, the mutant and ligand bind with an equilibrium binding constant K_{D}<10 μM. In one embodiment, the mutant and ligand bind with an equilibrium binding constant K_{D}<10 nM. In one embodiment, the mutant and ligand bind with an equilibrium binding constant K_{D}<100 pM. In one embodiment, the mutant and ligand bind with an equilibrium binding constant K_{D}<10 pM. In one embodiment, the mutant and ligand bind with an equilibrium binding constant K_{D}<100 nM. In one embodiment, the mutant and ligand bind with an equilibrium binding constant K_{D}<1 nM. All individual values and intermediate ranges of equilibrium binding constants less than 100 μM are included herein, including specifically for the purpose of use in the claims to exclude prior art.

Also provided is a soluble mutant T cell receptor (TCR) variable region having higher affinity than the wild type T cell receptor variable region for a bacterial superantigen, wherein said T cell receptor variable region is a mutant T cell receptor variable region carrying one or more mutations in a TCR variable region. In one embodiment, the TCR variable region exhibits an equilibrium binding constant K_{D} for the bacterial superantigen of between about 10⁻⁸M and 10⁻¹²M. In one embodiment, the TCR variable region is a mutant TCR having one or more mutations in a CDR. In one embodiment, the TCR variable region is a mutant TCR having one or more mutations in a FR region. In one embodiment, the bacterial superantigen is toxic shock syndrome toxin-1. In one embodiment, the bacterial superantigen is SEC. In one embodiment, the TCR variable region has one or more mutations in the human Vβ2 region. In one embodiment, the TCR variable region has one or more mutations in the Vβ2.1 region. In one embodiment, the TCR variable region has one or more mutations in CDR2. In one embodiment, the bacterial superantigen is staphylococcal enterotoxin B. In one embodiment, the TCR variable region has one or more mutations in the mouse Vβ8 domain. In one embodiment, the TCR variable region has one or more mutations in the Vβ8.2 domain. In one embodiment, the variable region is selected from Seq. ID Nos. 16-22; 30-44; and 66-73. In one embodiment, the TCR variable region is designated L3. In one embodiment, the TCR variable region is selected from Seq. ID Nos. 74-77.

Also provided is a method for treating staphylococcus infection in a mammal, the method comprising: providing an effective amount of a high affinity mutant TCR variable region having one or more mutations in the TCR variable beta region, which TCR variable region binds to the superantigen with higher affinity than wild type TCR variable region, wherein the high affinity TCR variable region interferes with the binding of the superantigen to the MHC class II molecules and T cell receptors of the mammal.

Also provided is a method of treating a disease state in a mammal caused by a bacterial superantigen comprising: administering an effective amount of a high affinity mutant of the T cell receptor variable region to a mammal. In one embodiment, the mammal is a human. In one embodiment, the variable region is a variable beta region. In one embodiment, the disease is selected from the group consisting of: pneumonia, mastitis, phlebitis, meningitis, urinary tract infections; osteomyelitis, endocarditis, nosocomial infection, staphylococcal food poisoning and toxic shock syndrome. In one embodiment, the T cell receptor variable region is selected from Seq. ID Nos. 16-22; 30-44; and 66-73. In one embodiment, the TCR variable region is selected from Seq. ID Nos. 74-77.

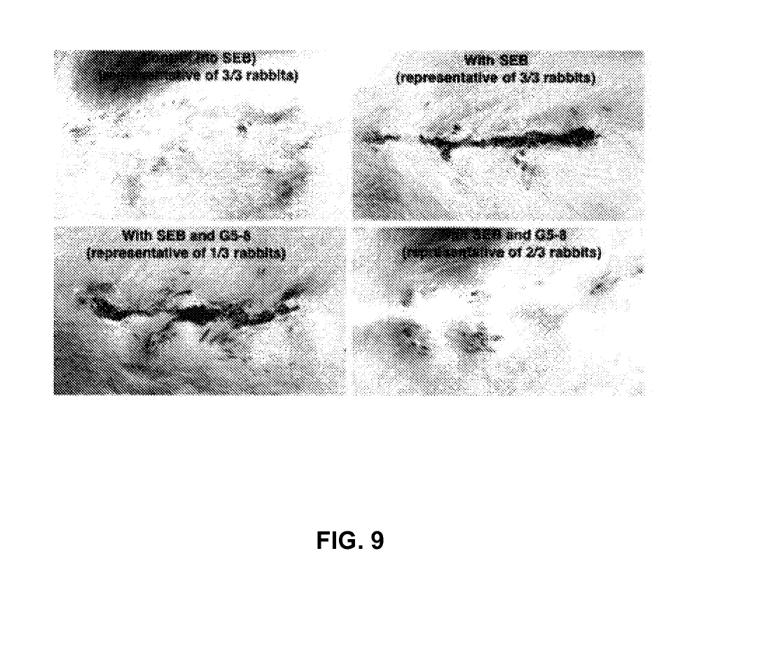

TABLE 1

| SEQ. ID NO. | SEQUENCE DESIGNATION IN FIG. 9 |
|---|---|
| 1 | WT |
| 2 | EP-5 |
| 3 | EP-6 |
| 4 | EP-7 |
| 5 | EP-8 |
| 6 | EP-9 |
| 7 | EP-11 |
| 8 | EP-12 |
| 9 | R3 |
| 10 | R9 |
| 11 | R15 |
| 12 | R17 |
| 13 | R18 |
| 14 | R21 |
| 15 | R24 |
| 16 | C4 |
| 17 | C8 |
| 18 | C10 |
| 19 | D9 |
| 20 | D10 |
| 21 | D19 |
| 22 | D20 |

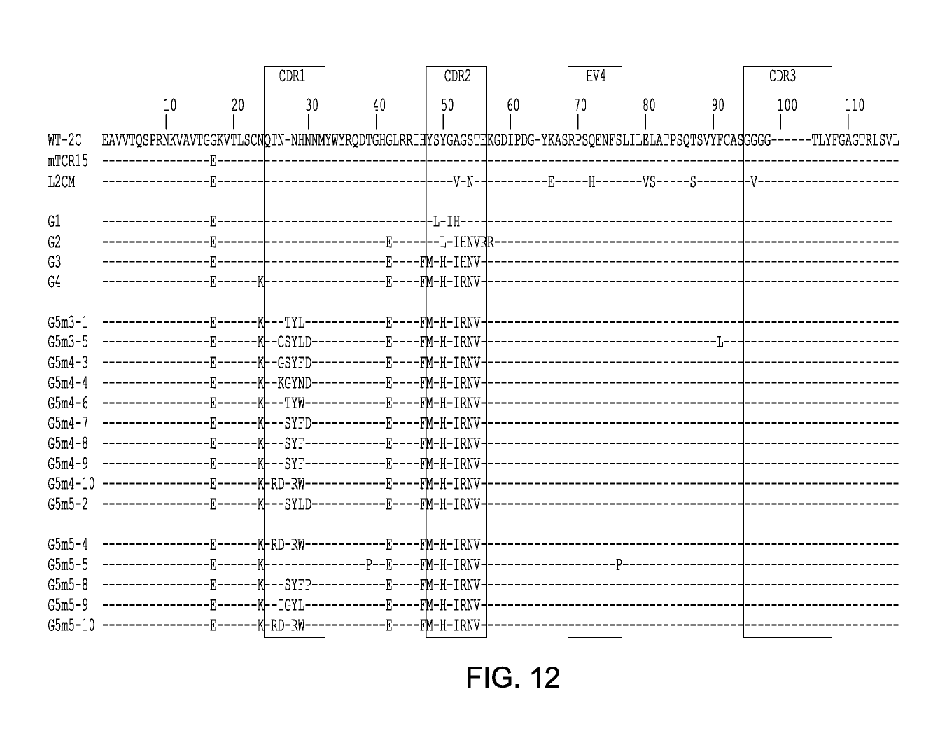

TABLE 2

| SEQ. ID NO. | SEQUENCE DESIGNATION IN FIG. 12 |
|---|---|
| 23 | WT-2C |
| 24 | mTCR15 |
| 25 | L2CM |
| 26 | G1 |
| 27 | G2 |
| 28 | G3 |
| 29 | G4 |
| 30 | G5m3-1 |
| 31 | G5m3-5 |
| 32 | G5m4-3 |
| 33 | G5m4-4 |
| 34 | G5m4-6 |
| 35 | G5m4-7 |
| 36 | G5m4-8 |
| 37 | G5m4-9 |
| 38 | G5m4-10 |
| 39 | G5m5-2 |
| 40 | G5m5-4 |
| 41 | G5m5-5 |
| 42 | G5m5-8 |
| 43 | G5m5-9 |
| 44 | G5m5-10 |

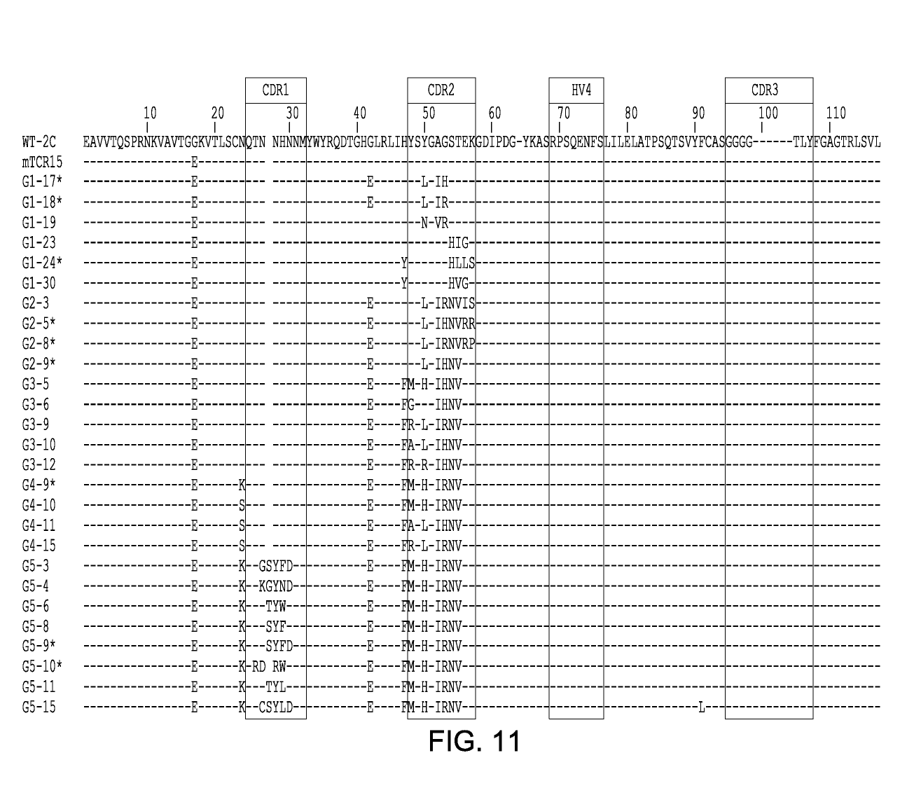

TABLE 3

| SEQ. ID NO. | SEQUENCE DESIGNATION IN FIG. 11 |
|---|---|
| 45 | WT-2c |
| 46 | mTCR15 |
| 47 | G1-17 |
| 48 | G1-18 |
| 49 | G1-19 |
| 50 | G1-23 |
| 51 | G1-24 |
| 52 | G1-30 |
| 53 | G2-3 |
| 54 | G2-5 |
| 55 | G2-8 |
| 56 | G2-9 |
| 57 | G3-5 |
| 58 | G3-6 |
| 59 | G3-9 |
| 60 | G3-10 |
| 61 | G3-12 |
| 62 | G4-9 |
| 63 | G4-10 |
| 64 | G4-11 |
| 65 | G4-15 |
| 66 | G5-3 |
| 67 | G5-4 |
| 68 | G5-6 |
| 69 | G5-8 |
| 70 | G5-9 |
| 71 | G5-10 |
| 72 | G5-11 |
| 73 | G5-15 |

Presented below is the wild type Vβ2.1 sequence before stabilization.

GAVVSQHPSRVIAKSGTSVKIECRSLDFQATTMFWYRQFPKQSLMLMATS
NEGSKATYEQGVEKDKFLINHASLTLSTLTVTSAHPEDSSFYICSALAGS
GSSTDTQYFGPGTRLTVL

Presented below is the wild type Vβ8.2 sequence designated WT-2c in FIG. 11.

EAVVTQSPRNKVAVTGGKVTLSCNQTN-
NHNNMYWYRQDTGHGLRLIHYSYGAGST
EKGDIPDG-YKASRPSQENFSLILELATPSQTSVYFCASGGGG------
TLYFGAGTRLSVL

Sequences of several embodiments of neutralizing agents are shown in Table 4. In embodiments, G5-8 neutralizes SEB and SpeA; L3 neutralizes SEC and SEB; D10 neutralizes TSST-1, and D10V, a double mutant of D10 neutralizes TSST-1.

Also provided are the Vbeta drugs comprising the sequences shown in Table 4.

TABLE 4

| SEQ. ID NO. | | Example | Key |
|---|---|---|---|
| (designation) | Sequence | toxins | mutations |
| 74 | GAVVSQHPSMVIVKSGTSVKIECRSLDTNIHTMFWY | TSST-1 | S52aF, |
| (D10) | RQFPKQSLMLMATSHQGFNAIYEQGVVKDKFLINH | | K53N, |
| | ASPTLSTLTVTSAHPEDSGFYVCSALAGSGSSTDTQ | | E61V |
| | YFGPGTQLTVL | | |
| 75 | GAVVSQHPSMVIVKSGTSVKIECRSLDTNIHTM | TSST-1 | Q51E, |
| (D10V) | FWYRQFPKQSLMLMATSHEGFNAIYEQGVTKD | | V61T |
| | KFLINHASPTLSTLTVTSAHPEDSGFYVCSALA | | |
| | GSGSSTDTQYFGPGTQLTVL | | |
| 76 | EAVVTQSPRNKVAVTGEKVTLSCKQTNSYFNN | SEB and | H28Y, |
| (G5-8) | MYWYRQDTGHELRLIFMSHGIRNVEKGDIPDG | SpeA | A52I, |
| | YKASRPSQENFSLILELATPSQTSVYFCASGGG | | G53R, |
| | GTLYFGAGTRLSVL | | S54N |
| 77 | EAVVTQSPRNKVAVTGEKVTLSCKQTNSYFDN | SEC | A52V, |
| (L3) | MYWYRQDTGHELRLIHYSYGVGNTEKGDIPDG | and SEB | S54N |
| | YEASRLTWRTFSLILVSATPSQTSVYFCASGGG | | |
| | GTLYFGAGTRLSVL | | |
| 78 | GAVVSQHPSRVIAKSGTSVKIECRSLDFQATTM | | |
| (Wildtype | FWYRQFPKQSLMLMATSNEGSKATYEQGVEK | | |
| Vb2) | DKFLINHASLTLSTLTVTSAHPEDSSFYICSALA | | |
| | GSGSSTDTQYFGPGTRLTVL | | |
| 79 | EAVVTQSPRNKVAVTGEKVTLSCNQTNNHNN | | |
| (Wildtype | MYWYRQDTGHGLRLIHYSYGAGSTEKGDIPDG | | |
| Vb8) | YKASRPSQENFSLILELATPSQTSVYFCASGGG | | |
| | GTLYFGAGTRLSVL | | |

In the sequences provided in Table 4, the underlined residues are different than the wild type. In an embodiment, the mutations provided in the right hand column of Table 4 are useful for high affinity. In an embodiment, the mutations provided in the right hand column of Table 4 are the minimum useful for high affinity. In a separate embodiment, there are other mutations that can be made that are useful for improving affinity or for other useful purposes. These modifications are easily determinable to one of ordinary skill in the art.

Also provided is a therapeutic composition comprising a stabilized T cell receptor variable region and optional pharmaceutical additives. Provided are compositions comprising soluble protein domains of the T cell receptor variable region that have high-affinity for a ligand, and methods for preparation thereof. In one embodiment, the ligand is a superantigen. The compositions bind to the active site of the superantigen and prevent or decrease the normal effect of the superantigen. These compositions are useful as therapeutics for those animals, including mammals, including humans, which are affected by a disease caused by the superantigen.

The compositions of the invention are prepared and selected using yeast display techniques described in detail elsewhere, in an embodiment. Generally, a library of mutants of the protein of interest are displayed on yeast cells and labeled with fluorescently labeled antibodies. The library is screened and those yeast cells displaying mutants which bind to the desired ligand with higher affinity are selected. The selected mutants can be mutagenized and screened for as many rounds as desired or required to provide the mutant with a desired affinity.

Regions and positions for site-directed mutagenesis of the T cell receptor variable region may be determined by selecting portions of the T cell receptor variable region that are believed to contact the superantigen (“contact regions”). These contact regions can be determined by structural models or calculations, as known in the art. For the systems described herein, the contact regions are primarily in the CDR2 and framework (FR) regions.

The compositions described herein are about 12,000 daltons in embodiments, although larger or smaller compositions are included in this invention and prepared by one of ordinary skill in the art without undue experimentation.

As used herein, a “stabilized” protein means the protein is displayable on yeast. As shown previously, wild type single-chain T cell receptor domains are not displayable on yeast, and require at least one mutation to display the properly folded protein. (PNAS 96:5651 (1999); J. Mol. Biol. 292:949 (1999); Nature Biotech. 18:754 (2000)). The mutation may be in any region or regions of the variable domain that results in a stabilized protein. In one embodiment, one or more mutations is in one or more of CDR1, CDR2, HV4, CDR3, FR2, and FR3. The regions used for mutagenesis can be determined by directed evolution, where crystal structures or molecular models are used to generate regions of the TCR which interact with the ligand of interest (toxin or antigen, for example). In other examples, the variable region can be reshaped, by adding or deleting amino acids to engineer a desired interaction between the variable region and the ligand.

The yeast display cloning vector used in these experiments can be any vector which allows insertion of the mutated protein and display on yeast. One particular example of a yeast display cloning vector is pCT202, which is shown in PCT/US 07/64085. The use of this vector has been described previously. The mutations that allow surface display also yield thermally stable, soluble variable region domains that can be secreted from yeast.

This invention provides a method for making stabilized T cell receptor (TCR) variable domains. These stabilized TCR variable domains are useful as receptor antagonists for ligands such as SEB, TSST-1, and SEC3. The methodology exemplified in the examples can be used to make stabilized TCR variable domains for any antigen. The terms “variable region” and “variable domain” are used interchangeably. “Vβ domain” refers to the variable region of a T cell receptor beta chain, or a fragment thereof, able to bind to a bacterial SAg. Currently, there are over 20 known VR domains able to bind to bacterial SAgs. As described herein, a polypeptide may be synthesized or expressed to contain one or more Vβ domains, or contain amino acid sequences functionally equivalent to one or more Vβ domains.

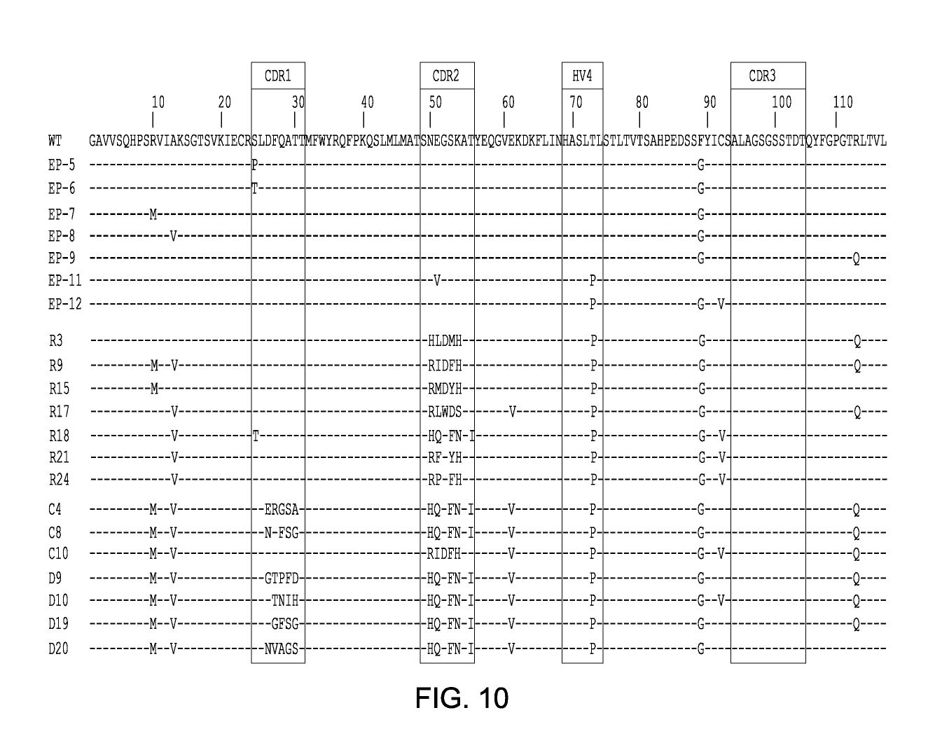

In one embodiment, stabilized proteins for TSST-1 are hVβ2.1 regions with one or more of the mutations S88G, RIM, A13V, L72P, and R113Q. In one embodiment, neutralizing agents for TSST-1 include those clones having the sequences exemplified with designations C4, C8, C10, D9, D10, D19, and D20 in FIG. 10. In one embodiment, neutralizing agents for TSST-1 have more than 5000 times increase in affinity for the toxin than the wild type. In one embodiment, stabilized proteins for SEB are mVβ8.2 regions with the mutation G17E and optionally one or more mutations selected from the group consisting of: N24K, G42E, H47F, Y48M, Y50H, A52I, G53R, S54N, and T55V. In one embodiment, neutralizing agents for SEB include those clones having the sequences exemplified with designations G5-x (x=3, 4, 6, 8, 9, 10, 11, 15) in FIG. 11. In one embodiment, neutralizing agents for SEB have more than 5000 times increase in affinity for the toxin than the wild type. All variable region sequences that are stabilized are individually included in this disclosure. All variable region sequences given here that have higher affinity for a ligand than a wild type sequence are individually included in this disclosure.

Therapeutic products can be made using the materials shown herein. Effective amounts of therapeutic products are the minimum dose that produces a measurable effect in a subject. Therapeutic products are easily prepared by one of ordinary skill in the art. In one embodiment, the variable domain is administered directly to a patient. In one embodiment, the variable domain is linked to an immunoglobulin constant region and used as a therapeutic. This embodiment extends the lifetime of the variable domain in the serum. In one embodiment, the variable domain is linked to PEG, as known in the art. This embodiment lengthens the serum clearance. These methods and other methods of administering, such as intravenously, are known in the art. Useful dosages are easily determined by one of ordinary skill in the art.

Mutagenesis methods used here include the use of mutator strains of E. coli, error-prone PCR, site-directed mutagenesis with degenerate primers/PCR, DNA shuffling, and other methods known in the art. Cloning methods used include standard ligations and electroporation, and homologous recombination of PCR products. Library sizes of up to 10⁷ molecules, for example, are formed. One method of analysis, fluorescent-activated cell sorting has been described previously. In an embodiment, the protein or sequence can be prepared using a recombinant method. In an embodiment, the protein or sequence can be prepared using a synthetic method.

In the methods for making neutralizing agents described herein, a stabilized T cell receptor variable region is used as the starting material for additional rounds of mutations and sorting. This process gives neutralizing agents with increasingly higher affinity to a toxin or antigen of interest. As used herein, “neutralizing agent” is a protein or protein fragment which binds to a molecule of interest with greater affinity than a wild type protein or protein fragment and is also referred to as “high affinity.” In one embodiment, the neutralizing agent has an affinity for the molecule of interest of more 5,000 times that of the wild type. In one embodiment, the neutralizing agent has an affinity for the molecule of interest of more 10,000 times that of the wild type. In one embodiment, the neutralizing agent has an affinity for the molecule of interest of more than 100,000 times that of wild type. Herein, the usage of the terms dissociation constant and equilibrium binding constant are consistent with the usage in the art and the context given.

In the figures and tables which present amino acid sequences, the wild type is designated “WT”. In the sequences presented below a top sequence, a dash indicates the amino acid is the same as the top sequence. A letter indicates a substitution has been made in that position from the top sequence.

In one embodiment of the invention, administration of an effective amount of a neutralizing agent is useful in preventing or reducing the toxic effects of a bacterial superantigen. In one embodiment of the invention, administration of an effective amount of a neutralizing agent prevents or reduces the binding of a bacterial superantigen to the variable region. In one embodiment of the invention, administration of an effective amount of a neutralizing agent prevents or reduces the crosslinking of the variable region and MHC.

Methods of this invention comprise the step of administering an “effective amount” of the compositions, formulations and preparations containing the described compounds or compositions to treat, reduce, alleviate, ameliorate or regulate a biological condition and/or disease state in a patient (also referred to as “treat” in general). The term “effective amount,” as used herein, refers to the amount of the diagnostic and therapeutic formulation, that, when administered to the individual is effective to treat, reduce alleviate, ameliorate or regulate a biological condition and/or disease state as described herein, for example a wound, pneumonia, or atopic dermatitis. As is understood in the art, an effective amount of a given composition or formulation will depend at least in part upon the mode of administration (e.g. intravenous, oral, topical administration), any carrier or vehicle employed, and the specific individual to whom the formulation is to be administered (age, weight, condition, sex, etc.). The dosage requirements needed to achieve the “effective amount” vary with the particular formulations employed, the route of administration, and clinical objectives. Based on the results obtained in standard pharmacological test procedures, projected daily dosages of active compound or composition can be determined as is understood in the art.

In an embodiment, an effective amount of a compound or composition of the invention is a therapeutically effective amount. As used herein, the phrase “therapeutically effective” qualifies the amount of compound or composition administered in the therapy. This amount achieves the goal of ameliorating, suppressing, eradicating, preventing, reducing the risk of, or delaying the onset of a targeted condition. In an embodiment, an active ingredient or other component is included in a therapeutically acceptable amount.

When used herein, the terms “treat”, “treatment” and other root word derivatives are as understood in the art and are further intended to include a general improvement in a state of health or disease such as reduce or regulate a biological condition and/or disease state in a patient. The term is not meant to necessarily encompass the concept of cure. For example, the treatment of pneumonia can include an improvement in symptom or a test result associated with the presence of a disease state of pneumonia. The term does not necessarily imply a defined level of certainty regarding the prediction of a particular status or outcome. The identified compounds treat, inhibit, control and/or prevent, or at least partially arrest or partially prevent, diseases and conditions of interest and can be administered to a subject at therapeutically effective amounts. In an embodiment, the term “treat” or other forms thereof is intended to encompass any measurable or detectable improvement of a condition or effect at least partially resulting from a bacterial superantigen or exotoxin. Compositions/formulations of the present invention comprise a therapeutically effective amount (which can optionally include a diagnostically effective amount) of at least one compound or bioconjugate of the present invention. Subjects receiving treatment that includes a compound/bioconjugate of the invention are preferably animals (e.g., mammals, reptiles and/or avians), more preferably humans, horses, cows, dogs, cats, sheep, pigs, and/or chickens, and most preferably humans.

As defined herein, “administering” means that a compound or formulation thereof of the invention, is provided to a patient or subject, for example in a therapeutically effective amount. The invention includes methods for a biomedical procedure wherein a therapeutically effective amount of a compound described herein is administered to a patient in need of treatment, for example to a patient undergoing treatment for a diagnosed diseased state. Administering can be carried out by a range of techniques known in the art including parenteral administration including intravenous, intraperitoneal or subcutaneous injection or infusion, oral administration, topical or transdermal absorption through the skin, or by inhalation, for example. The chosen route of administration may depend on such factors as solubility of the compound or composition, location of targeted condition, and other factors which are within the knowledge of one having ordinary skill in the relevant art.

“Topical administration” includes the use of transdermal administration, such as transdermal patches or iontophoresis devices.

“Parenteral administration” includes subcutaneous injections, intravenous injections, intraarterial injections, intraorbital injections, intracapsular injections, intraspinal injections, intraperitoneal injections, intramuscular injections, intrasternal injections, and infusion. Dosage forms suitable for parenteral administration include solutions, suspensions, dispersions, emulsions, and any other dosage form that can be administered parenterally.

As used herein, the term “controlled-release component” refers to an agent that facilitates the controlled-release of a compound including, but not limited to, polymers, polymer matrices, gels, permeable membranes, liposomes, microspheres, or the like, or any combination thereof. Methods for producing compounds in combination with controlled-release components are known to those of skill in the art.

As used herein, the term “pharmaceutically acceptable” means approved by a regulatory agency of an appropriate federal or state government; or listed in the U.S. Pharmacopoeia or other generally recognized pharmacopoeia for use in animals, and more particularly in humans; or does not impart significant deleterious or undesirable effect on a subject to whom it is administered and in the context in which it is administered.

It is contemplated that the compounds and pharmaceutically acceptable salts of the invention can be used as part of a combination. The term “combination” means the administration of two or more compounds directed to a target condition. The treatments of the combination generally can be co-administered in a simultaneous manner. Two compounds can be co-administered as, for example: (a) a single formulation (e.g., a single capsule) having a fixed ratio of active ingredients; or (b) multiple, separate formulations (e.g., multiple capsules) for each compound. The treatments of the combination can alternatively (or additionally) be administered at different times. In an embodiment, one compound is a conventional treatment, such as an antibiotic, and one compound is a drug of the invention.

In certain embodiments, the invention encompasses administering compounds useful in the invention to a patient or subject. A “patient” or “subject”, used equivalently herein, refers to an animal. In particular, an animal refers to a mammal, preferably a human. The subject can either: (1) have a condition able to prevented and/or treated by administration of a drug of the invention; or (2) is susceptible to a condition that is able to be prevented and/or treated by administering a drug of the invention.

The term “amino acid” comprises naturally occurring amino acids as well as non-naturally occurring amino acids, including amino acid analogs and derivatives. One skilled in the art will recognize that reference herein to an amino acid comprises, for example, naturally occurring proteogenic L-amino acids; D-amino acids; chemically modified amino acids such as amino acid analogs and derivatives; naturally occurring non-proteogenic amino acids, and chemically synthesized compounds having properties known in the art to be characteristic of amino acids.

The term “nucleic acid” as used herein generally refers to a molecule or strand of DNA, RNA, or derivatives or analogs thereof including one or more nucleobases. Nucleobases comprise purine or pyrimidine bases typically found in DNA or RNA (e.g., adenine, guanine, thymine, cytosine, and/or uracil). The term “nucleic acid” also comprises oligonucleotides and polynucleotides. Nucleic acids may be single-stranded molecules, or they may be double-, triple- or quadruple-stranded molecules that may comprise one or more complementary strands of a particular molecule. “Nucleic acid” includes artificial nucleic acids including peptide nucleic acids, morpholino nucleic acids, glycol nucleic acids and threose nucleic acids. Artificial nucleic acids may be capable of nucleic acid hybridization.

As used herein, “sequence” means the linear order in which monomers occur in a polymer, the order of amino acids in a polypeptide or the order of nucleotides in a polynucleotide for example.

The terms “peptide” and “polypeptide” are used synonymously in the present description, and refer to a class of compounds composed of amino acid residues chemically bonded together by amide bonds (or peptide bonds), regardless of length, functionality, environment, or associated molecule(s) Peptides and polypeptides are polymeric compounds comprising at least two amino acid residues or modified amino acid residues. Modifications can be naturally occurring or non-naturally occurring, such as modifications generated by chemical synthesis. Modifications to amino acids in peptides include, but are not limited to, phosphorylation, glycosylation, lipidation, prenylation, sulfonation, hydroxylation, acetylation, methionine oxidation, alkylation, acylation, carbamylation, iodination and the addition of cofactors. Peptides include proteins and further include compositions generated by degradation of proteins, for example by proteolyic digestion. Peptides and polypeptides can be generated by substantially complete digestion or by partial digestion of proteins. Polypeptides comprising 2 to 100 amino acid units, optionally for some embodiments 2 to 50 amino acid units and, optionally for some embodiments 2 to 20 amino acid units can be used as polypeptide targeting ligands in the invention, for example, where the polypeptide preferentially binds to proteins, peptides or other biomolecules expressed, or otherwise generated by, a target tissue, such as a tumor, precancerous tissue, site of inflammation or other lesion. Typically, the polypeptide is at least four amino acid residues in length and can range up to a full-length protein.

Provided below are specific examples of the invention which are to be considered to be non-limiting. The references described in bracketed numbers in each example are provided at the end of each example.

In order to provide a clear and consistent understanding of the specification and claims, including the scope to be given to such terms, the definitions herein are provided.

A coding sequence is the part of a gene or cDNA which codes for the amino acid sequence of a protein, or for a functional RNA such as a tRNA or rRNA.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′.

Downstream means on the 3′ side of any site in DNA or RNA.

Expression refers to the transcription of a gene into structural RNA (rRNA, tRNA) or messenger RNA (mRNA) and subsequent translation of a mRNA into a protein.

An amino acid sequence that is functionally equivalent to a specifically exemplified TCR sequence is an amino acid sequence that has been modified by single or multiple amino acid substitutions, by addition and/or deletion of amino acids, or where one or more amino acids have been chemically modified, but which nevertheless retains the binding specificity and high affinity binding activity of a cell-bound or a soluble TCR protein of the present invention. Functionally equivalent nucleotide sequences are those that encode polypeptides having substantially the same biological activity as a specifically exemplified cell-bound or soluble TCR protein. In the context of the present invention, a soluble TCR protein lacks the portions of a native cell-bound TCR and is stable in solution (i.e., it does not generally aggregate in solution when handled as described herein and under standard conditions for protein solutions).

Two nucleic acid sequences are heterologous to one another if the sequences are derived from separate organisms, whether or not such organisms are of different species, as long as the sequences do not naturally occur together in the same arrangement in the same organism.

Homology refers to the extent of identity between two nucleotide or amino acid sequences.

Isolated means altered by the hand of man from the natural state. If an “isolated” composition or substance occurs in nature, it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

A linker region is an amino acid sequence that operably links two functional or structural domains of a protein.

A nucleic acid construct is a nucleic acid molecule which is isolated from a naturally occurring gene or which has been modified to contain segments of nucleic acid which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

Two DNA sequences are operably linked if the nature of the linkage does not interfere with the ability of the sequences to affect their normal functions relative to each other. For instance, a promoter region would be operably linked to a coding sequence if the promoter were capable of effecting transcription of that coding sequence.

A polypeptide is a linear polymer of amino acids that are linked by peptide bonds.

Promoter means a cis-acting DNA sequence, generally 80-120 base pairs long and located upstream of the initiation site of a gene, to which RNA polymerase may bind and initiate correct transcription. There can be associated additional transcription regulatory sequences which provide on/off regulation of transcription and/or which enhance (increase) expression of the downstream coding sequence.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site).

Transformation means the directed modification of the genome of a cell by the external application of purified recombinant DNA from another cell of different genotype, leading to its uptake and integration into the subject cell=s genome. In bacteria, the recombinant DNA is not typically integrated into the bacterial chromosome, but instead replicates autonomously as a plasmid.

Upstream means on the 5′ side of any site in DNA or RNA.

A vector is a nucleic acid molecule that is able to replicate autonomously in a host cell and can accept foreign DNA. A vector carries its own origin of replication, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include plasmid vectors and phage vectors.

High affinity T cell receptor (TCR) means an engineered TCR with stronger binding to a target ligand than the wild type TCR. Some examples of high affinity include an equilibrium binding constant for a bacterial superantigen of between about 10-8 M and 10-12 M and all individual values and ranges therein.

It will be appreciated by those of skill in the art that, due to the degeneracy of the genetic code, numerous functionally equivalent nucleotide sequences encode the same amino acid sequence.

Additionally, those of skill in the art, through standard mutagenesis techniques, in conjunction with the assays described herein, can obtain altered TCR sequences and test them for the expression of polypeptides having particular binding affinity. Useful mutagenesis techniques known in the art include, without limitation, oligonucleotide-directed mutagenesis, region-specific mutagenesis, linker-scanning mutagenesis, and site-directed mutagenesis by PCR [see e.g. Sambrook et al. (1989) and Ausubel et al. (1999)].

In obtaining variant TCR coding sequences, those of ordinary skill in the art will recognize that TCR-derived proteins may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

Homology between nucleotide sequences can be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology. For instance, for sequences with about 55% G-C content, hybridization and wash conditions of 40-50° C., 6×SSC (sodium chloride/sodium citrate buffer) and 0.1% SDS (sodium dodecyl sulfate) indicate about 60-70% homology, hybridization and wash conditions of 50-65° C., 1×SSC and 0.1% SDS indicate about 82-97% homology, and hybridization and wash conditions of 52° C., 0.1×SSC and 0.1% SDS indicate about 99-100% homology. A wide range of computer programs for comparing nucleotide and amino acid sequences (and measuring the degree of homology) are also available, and a list providing sources of both commercially available and free software is found in Ausubel et al. (1999). Readily available sequence comparison and multiple sequence alignment algorithms are, respectively, the Basic Local Alignment Search Tool (BLAST) (Altschul et al., 1997) and ClustalW programs. BLAST is available on the Internet at http://www.ncbi.nlm.nih.gov and a version of ClustalW is available at http://www2.ebi.ac.uk.

Industrial strains of microorganisms (e.g., Aspergillus niger, Aspergillus ficuum, Aspergillus awamori, Aspergillus oryzae, Trichoderma reesei, Mucor miehei, Kluyveromyces lactis, Pichia pastoris, Saccharomyces cerevisiae, Escherichia coli, Bacillus subtilis or Bacillus licheniformis) or plant species (e.g., canola, soybean, corn, potato, barley, rye, wheat) may be used as host cells for the recombinant production of the TCR peptides. As the first step in the heterologous expression of a high affinity TCR protein or soluble protein, an expression construct is assembled to include the TCR or soluble TCR coding sequence and control sequences such as promoters, enhancers and terminators. Other sequences such as signal sequences and selectable markers may also be included. To achieve extracellular expression of the scTCR, the expression construct may include a secretory signal sequence. The signal sequence is not included on the expression construct if cytoplasmic expression is desired. The promoter and signal sequence are functional in the host cell and provide for expression and secretion of the TCR or soluble TCR protein. Transcriptional terminators are included to ensure efficient transcription. Ancillary sequences enhancing expression or protein purification may also be included in the expression construct.

Various promoters (transcriptional initiation regulatory region) may be used according to the invention. The selection of the appropriate promoter is dependent upon the proposed expression host. Promoters from heterologous sources may be used as long as they are functional in the chosen host.

Promoter selection is also dependent upon the desired efficiency and level of peptide or protein production. Inducible promoters such as tac are often employed in order to dramatically increase the level of protein expression in E. coli. Overexpression of proteins may be harmful to the host cells. Consequently, host cell growth may be limited. The use of inducible promoter systems allows the host cells to be cultivated to acceptable densities prior to induction of gene expression, thereby facilitating higher product yields.

Various signal sequences may be used according to the invention. A signal sequence which is homologous to the TCR coding sequence may be used. Alternatively, a signal sequence which has been selected or designed for efficient secretion and processing in the expression host may also be used. For example, suitable signal sequence/host cell pairs include the B. subtilis sacB signal sequence for secretion in B. subtilis, and the Saccharomyces cerevisiae α-mating factor or P. pastoris acid phosphatase phol signal sequences for P. pastoris secretion. The signal sequence may be joined directly through the sequence encoding the signal peptidase cleavage site to the protein coding sequence, or through a short nucleotide bridge consisting of usually fewer than ten codons, where the bridge ensures correct reading frame of the downstream TCR sequence.

Elements for enhancing transcription and translation have been identified for eukaryotic protein expression systems. For example, positioning the cauliflower mosaic virus (CaMV) promoter 1000 bp on either side of a heterologous promoter may elevate transcriptional levels by 10- to 400-fold in plant cells. The expression construct should also include the appropriate translational initiation sequences. Modification of the expression construct to include a Kozak consensus sequence for proper translational initiation may increase the level of translation by 10 fold.

A selective marker is often employed, which may be part of the expression construct or separate from it (e.g., carried by the expression vector), so that the marker may integrate at a site different from the gene of interest. Examples include markers that confer resistance to antibiotics (e.g., bla confers resistance to ampicillin for E. coli host cells, nptII confers kanamycin resistance to a wide variety of prokaryotic and eukaryotic cells) or that permit the host to grow on minimal medium (e.g., HIS4 enables P. pastoris or His⁻ S. cerevisiae to grow in the absence of histidine). The selectable marker has its own transcriptional and translational initiation and termination regulatory regions to allow for independent expression of the marker. If antibiotic resistance is employed as a marker, the concentration of the antibiotic for selection will vary depending upon the antibiotic, generally ranging from 10 to 600 μg of the antibiotic/mL of medium.

The expression construct is assembled by employing known recombinant DNA techniques (Sambrook et al., 1989; Ausubel et al., 1999). Restriction enzyme digestion and ligation are the basic steps employed to join two fragments of DNA. The ends of the DNA fragment may require modification prior to ligation, and this may be accomplished by filling in overhangs, deleting terminal portions of the fragment(s) with nucleases (e.g., ExoIII), site directed mutagenesis, or by adding new base pairs by PCR. Polylinkers and adaptors may be employed to facilitate joining of selected fragments. The expression construct is typically assembled in stages employing rounds of restriction, ligation, and transformation of E. coli. Numerous cloning vectors suitable for construction of the expression construct are known in the art (λZAP and pBLUESCRIPT SK-1, Stratagene, LaJolla, Calif.; pET, Novagen Inc., Madison, Wis.—cited in Ausubel et al., 1999) and the particular choice is not critical to the invention. The selection of cloning vector will be influenced by the gene transfer system selected for introduction of the expression construct into the host cell. At the end of each stage, the resulting construct may be analyzed by restriction, DNA sequence, hybridization and PCR analyses.

The expression construct may be transformed into the host as the cloning vector construct, either linear or circular, or may be removed from the cloning vector and used as is or introduced onto a delivery vector. The delivery vector facilitates the introduction and maintenance of the expression construct in the selected host cell type. The expression construct is introduced into the host cells by any of a number of known gene transfer systems (e.g., natural competence, chemically mediated transformation, protoplast transformation, electroporation, biolistic transformation, transfection, or conjugation) (Ausubel et al., 1999; Sambrook et al., 1989). The gene transfer system selected depends upon the host cells and vector systems used.

For instance, the expression construct can be introduced into S. cerevisiae cells by protoplast transformation or electroporation. Electroporation of S. cerevisiae is readily accomplished, and yields transformation efficiencies comparable to spheroplast transformation.

Monoclonal or polyclonal antibodies, preferably monoclonal, specifically reacting with a TCR protein at a site other than the ligand binding site may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1999) Current Protocols in Molecular Biology, John Wiley & Sons, Inc., New York.

High affinity TCR proteins in cell-bound or soluble form which are specific for a particular superantigen are useful, for example, as diagnostic probes for screening biological samples (such as cells, tissue samples, biopsy material, bodily fluids and the like) or for detecting the presence of the superantigen in a test sample. Frequently, the high affinity TCR proteins are labeled by joining, either covalently or noncovalently, a substance which provides a detectable signal. Suitable labels include but are not limited to radionuclides, enzymes, substrates, cofactors, inhibitors, fluorescent agents, chemiluminescent agents, magnetic particles and the like. Additionally the TCR protein can be coupled to a ligand for a second binding molecules: for example, the TCR protein can be biotinylated. Detection of the TCR bound to a target cell or molecule can then be effected by binding of a detectable streptavidin (a streptavidin to which a fluorescent, radioactive, chemiluminescent, or other detectable molecule is attached or to which an enzyme for which there is a chromophoric substrate available). United States patents describing the use of such labels and/or toxic compounds to be covalently bound to the scTCR protein include but are not limited to U.S. Pat. Nos. 3,817,837; 3,850,752; 3,927,193; 3,939,350; 3,996,345; 4,277,437; 4,275,149; 4,331,647; 4,348,376; 4,361,544; 4,468,457; 4,444,744; 4,640,561; 4,366,241; RE 35,500; 5,299,253; 5,101,827; 5,059,413. Labeled TCR proteins can be detected using a monitoring device or method appropriate to the label used. Fluorescence microscopy or fluorescence activated cell sorting can be used where the label is a fluorescent moiety, and where the label is a radionuclide, gamma counting, autoradiography or liquid scintillation counting, for example, can be used with the proviso that the method is appropriate to the sample being analyzed and the radionuclide used. In addition, there can be secondary detection molecules or particle employed where there is a detectable molecule or particle which recognized the portion of the TCR protein which is not part of the binding site for the superantigen or other ligand in the absence of a MHC component as noted herein. The art knows useful compounds for diagnostic imaging in situ; see, e.g., U.S. Pat. Nos. 5,101,827; 5,059,413. Radionuclides useful for therapy and/or imaging in vivo include ¹¹¹Indium, ⁹⁷Rubidium, ¹²⁵Iodine, ¹³¹Iodine, ¹²³Iodine, ⁶⁷Gallium, ⁹⁹Technetium. Toxins include diphtheria toxin, ricin and castor bean toxin, among others, with the proviso that once the TCR-toxin complex is bound to the cell, the toxic moiety is internalized so that it can exert its cytotoxic effect. Immunotoxin technology is well known to the art, and suitable toxic molecules include, without limitation, chemotherapeutic drugs such as vindesine, antifolates, e.g. methotrexate, cisplatin, mitomycin, .anthrocyclines such as daunomycin, daunorubicin or adriamycin, and cytotoxic proteins such as ribosome inactivating proteins (e.g., diphtheria toxin, pokeweed antiviral protein, abrin, ricin, pseudomonas exotoxin A or their recombinant derivatives. See, generally, e.g., Olsnes and Pihl (1982) Pharmac. Ther. 25:355-381 and Monoclonal Antibodies for Cancer Detection and Therapy, Eds. Baldwin and Byers, pp. 159-179, Academic Press, 1985.

High affinity TCR variable regions specific for a particular superantigen are useful in treating animals and mammals, including humans believed to be suffering from a disease associated with the particular superantigen.

The high affinity TCR variable region compositions can be formulated by any of the means known in the art. They can be typically prepared as injectables, especially for intravenous, intraperitoneal or synovial administration (with the route determined by the particular disease) or as formulations for intranasal or oral administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active ingredients are often mixed with optional pharmaceutical additives such as excipients or carriers which are pharmaceutically acceptable and compatible with the active ingredient. Suitable excipients include but are not limited to water, saline, dextrose, glycerol, ethanol, or the like and combinations thereof. The concentration of the TCR variable region in injectable, aerosol or nasal formulations is usually in the range of 0.05 to 5 mg/ml. The selection of the particular effective dosages is known and performed without undue experimentation by one of ordinary skill in the art. Similar dosages can be administered to other mucosal surfaces.

In addition, if desired, vaccines may contain minor amounts of pharmaceutical additives such as auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1-2′-dipalmitoyl-sn-glycero-3hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. Such additional formulations and modes of administration as are known in the art may also be used.

The TCR variable regions of the present invention and/or binding fragments having primary structure similar (more than 90% identity) to the TCR variable regions and which maintain the high affinity for the superantigen may be formulated into vaccines as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

TCR variable regions for therapeutic use are administered in a manner compatible with the dosage formulation, and in such amount and manner as are prophylactically and/or therapeutically effective, according to what is known to the art. The quantity to be administered, which is generally in the range of about 100 to 20,000 μg of protein per dose, more generally in the range of about 1000 to 10,000 μg of protein per dose. Similar compositions can be administered in similar ways using labeled TCR variable regions for use in imaging, for example, to detect cells to which a superantigen is bound. Precise amounts of the active ingredient required to be administered may depend on the judgment of the physician or veterinarian and may be peculiar to each individual, but such a determination is within the skill of such a practitioner.

The vaccine or other immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the retrovirus-like particles of the present invention are protected from infection by the cognate retrovirus.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

TSST-1 interacts almost exclusively with the human Vβ2.1 (hVβ2.1) region and a significant fraction of patients with TSS exhibit expansions of T cells with hVβ2.1. The structure of hVβ2.1 in complex with SpeC showed that hVβ2.1 uses a greater number of hypervariable regions for contact, compared to the interaction of mouse Vβ8.2 with its three different SAg ligands. Thus, residues from all three complementarity determining regions (CDRs) and hypervariable loop 4 (HV4) contributed contacts with SpeC and the interface exhibited a greater buried surface area than mVβ8.2-SAg interfaces. While the structure of the hVβ2.1-TSST-1 complex has not been solved, a recent alanine mutagenesis study of TSST-1 revealed the key residues of TSST-1 that are involved in the interaction.

Yeast display techniques were used to engineer the TCR for higher affinity binding to the desired superantigen. These yeast display techniques are described in U.S. Pat. Nos. 6,759,243; 6,696,251; 6,423,538; 6,300,065; 6,699,658, which are incorporated by reference to the extent not inconsistent with the disclosure herewith.

### Example 1

Staphylococcal toxic shock syndrome (TSS) has occasionally been reported without rash and desquamation. This study describes a patient who met all criteria for TSS except erythroderma and desquamation; the associated staphylococcal superantigen was enterotoxin B. We demonstrate erythroderma depends on pre-existing T cell hypersensitivity amplified by superantigenicity.

Staphylococcal toxic shock syndrome (TSS) is defined by fever, hypotension, erythroderma, desquamation, and variable multiorgan components [1]. Menstrual TSS occurs primarily in women using tampons [2] and is associated with the superantigen TSS toxin-1 (TSST-1) [3]. Non-menstrual TSS occurs in males and females initiated by any type of infection [4]; cases are associated with TSST-1 and staphylococcal enterotoxins B (SEB) and C [3].

When TSS was identified, it was recognized that cases occur where one defining criterion is absent; these cases are defined as probable TSS [4]. However, little attention has been paid to cases where multiple criteria are absent. Parsonnet suggests these cases be identified as toxin-mediated disease [5].

We describe a TSS patient whose initial diagnosis was difficult because erythroderma and desquamation were absent. Once identified as variant illness, the patient responded to intravenous immunoglobulin (IVIG) and clindamycin. We demonstrate that erythroderma depends on delayed hypersensitivity amplified by superantigenicity.

### Materials and Methods.

The patient's S. aureus was tested by PCR for genes for superantigens and Panton-Valentine leukocidin (PVL) [6], and by quantitative antibody assay for SEB after growth (Todd Hewitt broth; Becton, Dickinson and Company, Sparks, Md.) [7]. Positive and negative control strains responded as expected in both tests. Patient serum was tested by ELISA for SEB antibodies prior to administration of IVIG.

Eight Dutch-belted rabbits were tested with 1 ug/0.1 ml highly purified SEB intradermally on their flanks, and were monitored for 48 hr for erythroderma. The same animals were then given a subcutaneous sensitizing dose of SEB (25 ug) in Freund's incomplete adjuvant (Difco, Detroit, Mich.). After 2 weeks, the rabbits were injected intradermally with SEB (1 ug/0.1 ml), or SEB pre-mixed for 30 min with 10 ug soluble high-affinity variable region, β-chain T cell receptor (Vβ-TCR designated G5-8) capable of neutralizing superantigenicity [8]; animals were observed for erythroderma.

### Case Report.

A 16 year-old otherwise healthy teenager presented to urgent care with complaints of headache, deep breathing, and nausea for three days and increasing diffuse weakness for 24 hr. She denied fever, recent weight loss, vomiting, diarrhea, polyuria, or polydipsia. Past history revealed no illnesses, and she was not on medications. She began menses at age 11, described her cycles as “irregular”, and denied tampon use.

Her temperature was 95.5° F., blood pressure 119/80, pulse 117, respirations 28, with 100% oxygen saturation on room air. She was awake, in no acute distress, and cooperative. Her examination was notable for clear lungs with Kussmaul respirations and a 3-4 second capillary refill. No erythroderma was seen. The remainder of the exam was normal.

Initial laboratory data revealed metabolic acidosis (pH 6.77, pCO2 11, paO2 168, HCO3 2), with hyperglycemia (glucose level, 524 mg/dL). She was diagnosed with diabetic ketoacidosis (DKA) and treated with IV fluids and insulin. Her total white cell count was 7.6×109/L with differential of 82% neutrophils, 8% lymphocytes, and 9% monocytes. Hemoglobin was 15.9 mg/dL, and platelets numbered 158,000. Urinalysis demonstrated glucose of 300 mg/dL, ketones of 10 mg/dL, 14 red blood cells, 1 white blood cell, few bacteria, and amorphous crystals. A foley catheter was inserted to monitor urine output.

She was transferred to the pediatric intensive care unit (PICU). Three hours later, her condition worsened. She became combative and confused. Her serum osmolarity was 314, and IV mannitol was administered with resolution of her mental status changes. Shortly thereafter, she had a fever of 38.5° C. No antibiotics were started, and no cultures obtained. After 18 hr in the PICU, she again became combative and confused. Mannitol and bicarbonate were given without effect. Central access was obtained with a femoral venous catheter, and she was intubated. Subsequently, she became hypotensive (blood pressure 77/39) and was started on a norepinephrine drip. After 9 hr, she developed fever to 40.0° C. Blood, endotracheal, and urine cultures were obtained, and IV cefotaxime and vancomycin were started. Computed tomography scan revealed no cerebral edema.

She developed thrombocytopenia (platelets 25,000/mm³), acute renal failure (creatinine 1.98 mg/dL), and persistent hypotension requiring phenylephrine, norepinephrine, and dopamine 24 hr after admission. Further investigation showed negative serum toxicology screen, absence of serum salicylate and acetaminophen, normal thyroid studies, and echocardiogram and electrocardiogram with no significant abnormalities. The following were elevated: CK (833 mg/dL), lipase (371 U/L), amylase (738 U/L), and alanine transaminase (103 U/L). Her hemoglobin A1C level was 15.5%. Chest x-ray revealed no abnormalities. Due to sepsis concern, caspofungin and meropenem were started; cefotaxime was discontinued.

On day 3 of hospitalization, an endotracheal tube sputum culture taken shortly after intubation grew methicillin-sensitive S. aureus (MSSA), a urine culture grew 10,000-50,000 MSSA, and the next day the initial blood culture grew MSSA. TSS was suspected despite the lack of erythroderma. Clindamycin and cefazolin were started, and IVIG was given (1 gm/kg). Other antimicrobials were stopped. Further examination revealed no vaginal foreign objects, and repeat ECHO demonstrated no vegetations or regurgitation. She lacked mucosal hyperemia during hospitalization.

Within 6 hr of receiving IVIG and 9 hr of clindamycin, she was weaned off phenylephrine; within 12 hr, the norepinephrine drip was being weaned. Three days after receiving IVIG and clindamycin, her hypotension resolved, she was extubated to room air, and her mental status returned to baseline. Blood cultures taken over 4 days after initial blood culture were negative. A total of 5 days of clindamycin and 2 weeks of cefazolin were given. A 0.5 cm blister was noted on her right thumb 6 days after admission, but no other desquamation was noted, including the 1-2 weeks specified in the TSS definition.

### Superantigen Studies.

Her S. aureus contained genes for SEB and SE-like G, K, L, and N but not PVL. The strain produced SEB (77 ug/ml) in vitro; ELISA demonstrated low SEB antibodies titers (≦1:40, compared to 1:640 for IVIG). Approximately 10% of S. aureus strains produce SEB, and 90% of 15-20 year-old females should have titers >1:40 to SEB [9].

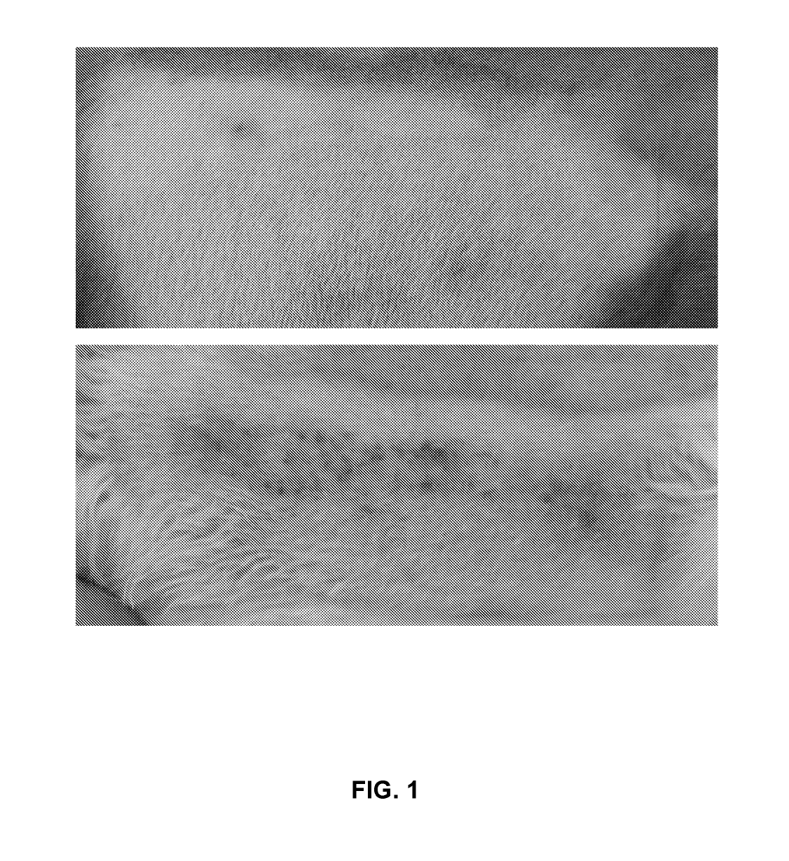

Eight rabbits were skin-tested with 1 ug/0.1 ml of SEB; none showed erythroderma. The same animals were sensitized to SEB for 2 weeks and then re-tested. Four rabbits received SEB and showed erythroderma (diameters 11±1.3 cm; example in FIG. 1). The remaining four rabbits received SEB pre-mixed with 10 ug soluble Vβ-TCR. These animals showed minor erythroderma (0.2±0.3 cm; example in FIG. 1) (p<<0.001 compared to treatment with SEB by Student's t test).

### Discussion.

TSS includes the characteristic erythroderma, fever, hypotension, desquamation, and multisystem organ involvement [1, 2]. A probable diagnosis can be made with one criterion absent. Our patient lacked erythroderma and desquamation, and thus did not meet the criteria for TSS or probable TSS. A small blister on her right hand was not felt to meet criteria for desquamation. However, the patient met all other criteria for TSS. No pathogens other than S. aureus were grown, and the patient's isolate produced SEB, a superantigen associated with non-menstrual TSS [3]. Finally, the patient's serum prior to IVIG therapy contained low SEB antibodies, so the patient was serosusceptible.

Because the patient did not have erythroderma, her clinical presentation was initially puzzling. Without initial fever, it is unclear whether S. aureus triggered DKA, or whether she became superinfected while in DKA. Her symptoms progressed to septic shock, but without the characteristic rash, it was difficult to implicate staphylococcal TSS; although it had been considered. With positive cultures, however, the diagnosis became evident.

The dramatic improvement after IVIG and clindamycin suggests a response to these treatments. Although we cannot know with certainty if clinical improvement was related to the treatments, anecdotal case reports and in vitro studies suggest IVIG and clindamycin may be effective therapies for TSS. Further data on their efficacy is required before they can be accepted as standard of care.

There are few full case reports of staphylococcal TSS without rash. Although early epidemiologic reports note a few cases lacking rashes [4], Van Lierde et al. [10] and Matsuda et al. [11] published full clinical descriptions of individual cases. Kamel et al. concluded that the absence of rash in three TSS patients, who underwent chemotherapy for multiple myeloma, reflected T cell deficiency [12].

An alternative explanation for the absence of erythroderma is that it results from delayed hypersensitivity amplified by superantigenicity. Some women describe partial episodes of menstrual TSS symptoms preceding definite TSS. In those prior episodes, erythroderma and desquamation may be absent, which is consistent with the need for hypersensitivity. The isolation of SEB from our patient's S. aureus strain indicates the illness did not originate from vaginal colonization. It is more likely that this is the patient's first encounter at a non-vaginal site with an SEB⁺ organism.

The rabbit experiments demonstrate erythroderma is associated with delayed hypersensitivity; the skin tests peaked in 24-48 hr. The studies with co-administration of SEB-neutralizing, high-affinity Vβ-TCRs (G5-8) demonstrate that superantigenicity is also required [8].

The present case highlights that TSS may present without erythroderma and desquamation, with cases more frequent than presently recognized. The case also highlights the need for further studies on whether IVIG and clindamycin decrease morbidity and mortality.

TABLE 5
Staphylococcal Toxic Shock Syndrome: Clinical Case Definition

| 1) | Fever: temperature greater than or equal to 38.9 C. (102 F.) | |
|---|---|---|
| 2) | Rash: diffuse macular erythroderma | |
| 3) | Desquamation: 1-2 weeks after onset, particularly palms, fingers and | |
| | toes | |
| 4) | Hypotension: systolic pressure less than or equal to 90 mmHg for | |
| | adults; lower than the fifth percentile for age for children younger | |
| | than 16 years of age; orthostatic drop in diastolic pressure of greater | |
| | than 15 mmHg from lying to sitting; orthostatic syncope or dizziness | |
| 5) | Negative results on the following tests if obtained: | |
| | a. | Blood, throat, or cerebrospinal fluid cultures; blood |
| | | culture may be positive for Staphylococcal aureus |
| | b. | Serologic tests for rocky Mountain spotted fever, leptospirosis, |
| | | or measles |
| 6) | Multisystem organ involvement: 3 or more of the following: | |
| | a. | Gastrointestinal: vomiting or diarrhea at onset of the illness |
| | b. | Muscular: severe myalgia or creatinine phosphokinase |
| | | concentration greater than twice the upper limit of normal |
| | c. | Mucous membrane: vaginal, oropharyngeal or conjunctival |
| | | hyperemia |
| | d. | Renal: serum urea nitrogen or creatinine concentration greater |
| | | than twice the upper limit of normal or urinary sediment with |
| | | greater than or equal to 5 WBC per HPF in the absence of |
| | | urinary tract infection |
| | e. | Hepatic: total bilirubin, aspartate or alanine transaminase |
| | | concentration greater than twice the upper limit of normal |
| | f. | Hematologic: platelet count less than or equal to 100,000/mm3 |
| | g. | Central nervous system: disorientation or alterations in |
| | | consciousness without focal neurological signs when fever and |
| | | hypotension are absent |

### Case Classification

Probable: ≧3 criteria plus desquamation, or ≧5 criteria without desquamation.
Confirmed: all 6 criteria, including desquamation.
Adapted from [1].

TABLE 6
Superantigen-induced erythroderma in rabbits depends on pre-existent
delayed hypersensitivity amplified by superantigenicity.

| Sensitizing Agent | Skin Test challenge agent | Skin test erythema diameter |
|---|---|---|
| None | SEB | 0 |
| SEB | SEB | 11 ± 1.3 |
| SEB | SEB + soluble Vβ-TCR | 0.2 ± 0.3 |

Note:
Sensitizing agent was 25 ug SEB emulsified in Freund's incomplete adjuvant.

Skin test reaction diameters were recorded after 24 hr. Skin reactions from sensitized SEB groups without and with G5-8 soluble high affinity Vβ-TCR differed by p<<0.001 by unpaired Student's t test of normally distributed.

### REFERENCES

1. Toxic Shock Syndrome. Red Book: Report of the Committee on Infectious Diseases. In: Pickering L K, Baker, C. J., Long, S. S. and McMillan, J. A., ed. Vol. 27. Elk Grove Village, Ill.: American Academy of Pediatrics, 2006:662
2. Shands K N, Schmid G P, Dan B B, et al. Toxic-shock syndrome in menstruating women: association with tampon use and Staphylococcus aureus and clinical features in 52 cases. N Engl J Med 1980; 303:1436-42
3. Davis J P, Chesney P J, Wand P J and LaVenture M. Toxic-shock syndrome: epidemiologic features, recurrence, risk factors, and prevention. N Engl J Med 1980; 303:1429-35
4. Reingold A L, Hargrett N T, Dan B B, Shands K N, Strickland B Y and Broome C V. Nonmenstrual toxic shock syndrome: a review of 130 cases. Ann Intern Med 1982; 96:871-4
5. Bergdoll M S, Crass B A, Reiser R F, Robbins R N and Davis J P. A new staphylococcal enterotoxin, enterotoxin F, associated with toxic-shock-syndrome Staphylococcus aureus isolates. Lancet 1981; 1:1017-21
6. Schlievert P M, Shands K N, Dan B B, Schmid G P and Nishimura R D. Identification and characterization of an exotoxin from Staphylococcus aureus associated with toxic-shock syndrome. J Infect Dis 1981; 143:509-16
7. Crass B A, Bergdoll M S. Involvement of staphylococcal enterotoxins in nonmenstrual toxic shock syndrome. J Clin Microbiol 1986; 23:1138-9.
8. McCormick J K, Yarwood J M and Schlievert P M. Toxic shock syndrome and bacterial superantigens: an update. Annu Rev Microbiol 2001; 55:77-104
9. Schlievert P M. Staphylococcal enterotoxin B and toxic-shock syndrome toxin-1 are significantly associated with non-menstrual TSS. Lancet 1986; 1:1149-50
10. Parsonnet J. Case definition of staphylococcal TSS: a proposed revision incorporating laboratory findings. International Congress and Symposium Series 1998; 229:15
11. Schlievert P M, Case L C. Molecular analysis of staphylococcal superantigens. Methods Mol Bioi 2007; 391: 113-26
12. Schlievert P M. Immunochemical assays for toxic shock syndrome toxin-i. Methods Enzymol 1988; 165:339-44
13. Blomster-Hautamaa D A, Schlievert P M. Preparation of toxic shock syndrome toxin-1. Methods Enzymol 1988; 165:37-43
14. Yang X, Buonpane R A, Moza B, et al. Neutralization of multiple staphylococcal superantigens by a single-chain protein consisting of affinity-matured, variable domain repeats. J Infect Dis 2008; 198:344-8
15. Buonpane R A, Churchill H R, Moza B, et al. Neutralization of staphylococcal enterotoxin B by soluble, high-affinity receptor antagonists. Nat Med 2007; 13:725-9
16. Schroder E, Kunstmann G, Hasbach Hand Pulverer G. Prevalence of serum antibodies to toxic-shock-syndrome-toxin-1 and to staphylococcal enterotoxins A, Band C in West-Germany. Zentralbl Bakteriol Mikrobiol Hyg [A] 1988; 270:110-4
17. Schlievert P M, Case L C, Strandberg K L, Tripp T J, Lin Y C and Peterson M L. Vaginal Staphylococcus aureus superantigen profile shift from 1980 and 1981 to 2003, 2004, and 2005. J Clin Microbiol 2007; 45:2704-7
18. Schlievert P M. Use of intravenous immunoglobulin in the treatment of staphylococcal and streptococcal toxic shock syndromes and related illnesses. J Allergy Clin Immunol 2001; 108:107 S-10S.
19. Schlievert P M, Kelly J A. Clindamycin-induced suppression of toxic-shock syndrome-1-associated exotoxin production. J Infect Dis 1984; 149:471
20. Van Lierde S, van Leeuwen W J, Ceuppens J, Cornette L, Goubau P and Van Eldere J. Toxic shock syndrome without rash in a young child: link with syndrome of hemorrhagic shock and encephalopathy? J Pediatr 1997; 131:130-4
21. Matsuda Y, Kato H, Yamada R, et al. Early and definitive diagnosis of toxic shock syndrome by detection of marked expansion of T-cell-receptor VBeta2-positive T cells. Emerg Infect Dis 2003; 9:387-9
22. Kamel N S, Banks M C, Dosik A, Ursea 0, Yarilina A A and Posnett O N. Lack of muco-cutaneous signs of toxic shock syndrome when T cells are absent: S. aureus shock in immunodeficient adults with multiple myeloma. Clin Exp Immunol 2002; 128:131-9
23. Schlievert P M, Bettin K M and Watson O W. Reinterpretation of the Dick test: role of group A streptococcal pyrogenic exotoxin. Infect Immun 1979; 26:467-72

### Example 2

### Staphylococcal Superantigens Cause Lethal Pulmonary Disease in Rabbits

### Background.

Recently, the CDC and others reported that methicillin-resistant S. aureus (MRSA) are the most significant causes of serious human infections in the United States, including pulmonary illnesses. We investigated the role of staphylococcal superantigens (SAgs) in lung-associated lethal infections in rabbits.

### Methods.

A rabbit model of lethal pulmonary disease was established in rabbits to investigate the potential role of SAgs, staphylococcal enterotoxin (SE) serotypes Band SEC, and toxic shock syndrome toxin-1 (TSST-1). Rabbits received intra-bronchial community-associated (CA) MRSA strains MNPA (TSST-1+), MW2 (SEC+), or c99-529 (SEB+), or purified SAgs. Animals were monitored for lethality for up to seven days. Some rabbits were pre-immunized against highly purified SAgs or treated with soluble high-affinity T cell receptors (Vβ-TCR) capable of neutralizing SEB and then also challenged intra-bronchially with CA-MRSA or SAgs.

### Results.

Rabbits challenged with viable CA-MRSA or purified SAgs developed fatal, pulmonary diseases. Animals pre-immunized against purified SAgs, or treated with soluble V13-TCRs, and then challenged with either viable bacteria or purified SAgs survived. Lung histology indicated non-immune animals developed hemorrhagic lesions when animals were challenged either with CA-MRSA or purified SAgs; SAg immune animals or animals treated with soluble Vβ-TCRs did not develop pulmonary lesions.

### Conclusions.

Staphylococcal SAgs contribute significantly to lethal pulmonary illnesses due to CA-MRSA; pre-existing immunity to SAgs prevents lethality. Administration of soluble high-affinity Vβ-TCR with specificity for SEB to non-immune animals protects from lethal pulmonary diseases due to SEB+ CA-MRSA and SEB.

### Introduction

Staphylococcus aureus is a significant human pathogen that causes multiple illnesses [1]. In recent years, there has been a rapid emergence of severe soft tissue and pulmonary infections caused by community-associated methicillin-resistant S. aureus (CA-MRSA) [2, 3]. These potentially fatal infections, including toxic shock syndrome (TSS), purpura fulminans, and necrotizing pneumonia occur in individuals lacking predisposing risk factors, although the majority may have had prior upper respiratory viral infections [2-4].

Staphylococcal superantigens (SAgs) are exotoxins that stimulate massive cytokine production by both T lymphocytes and macrophages [5, 6]. These cytokines include TNF-a and β, IL-1β, IL-2, and IFN-γ [7], and cause many of the clinical features of TSS. SAgs bind to and crosslink variable regions of certain β-chains of T cell receptors (Vβ-TCRs) and either or both of the α- or β-chains of major histocompatibility complex (MHC) II molecules on macrophages [8, 9].

SAgs, such as TSS toxin-1 (TSST-1) made by CA-MRSA USA200 strains (CDC designation based on pulsed-field gel electrophoresis) and staphylococcal enterotoxins (SEs) Band C made by CA-MRSA USA400 strains [4], are associated with TSS and purpura fulminans in humans [5, 6]. TSST-1 is associated with nearly all cases of menstrual TSS (mTSS), and 50% of non-menstrual cases. SEB and SEC are associated with most of the remaining cases of non-menstrual TSS [10, 11].

In the present study, we investigated the role of these three SAgs produced by CA-MRSA in rabbit models of pulmonary disease.

### Materials and Methods

### CA-MRSA Strains.

USA200 strains included MNPA, MN1 021, and MN128. These isolates produce TSST-1, but not α, β, γ, or Panton-Valentine leukocidin (PVL) cytotoxins. The strains have mutations in the a- and v-toxin genes as determined by nucleotide sequencing, lack PVL genes as determined by PCR, and have inactivated β-toxin genes [12, 13]. USA400 strains were MW2 and c99-529 [4]. MW2 produces SEC, while CA-MRSA c99-529 produces SEB. Both of the USA400 strains also produce a- and v-toxins and PVL, but not β-toxin.

### Rabbits.

Dutch belted rabbits (1.5 to 2 kg) were used in accordance with guidelines established by the University of Minnesota IACUC.

### Superantigens.

All reagents used for preparation of purified SAgs were maintained lipopolysaccharide (LPS)-free. TSST-1 was purified to homogeneity from S. aureus clone RN4220 (pCE107); this strain does not produce other SAgs. SEB was purified from S. aureus MNHo and SEC from MW2. SAgs were purified after growth of organisms in dialyzable beef heart media [14]. SAgs were precipitated from culture fluids with four volumes of absolute ethanol, resolubilized in distilled water, and purified by thin-layer isoelectric focusing [14, 15]. Initial isoelectric focusing pH gradients were 3.5-10, followed by second gradients of pH 6-8 for TSST-1 and pH 7-9 for SEB and SEC; the isoelectric point for TSST-1 is 7.2 and for SEB and SEC is 8.3 [16, 17]. Purified SAgs were quantified using the BioRad Protein assay (BioRad Co., Hercules, Calif.), with SEB as the protein standard. Purity was confirmed by SAg migration as single bands when subjected to sodium dodecyl sulfate polyacrylamide gel electrophoresis [18] with silver staining and reversed phase HPLC [19] [also confirming lack of contaminating lipoteichoic acid], Limulus assay to confirm lack of detectable LPS, and bioassays to confirm absence of detectable cytolysins, lipase, and protease [20]. Purified SAgs were free of detectable peptidoglycan and LPS also as demonstrated by lack of pyrogenicity with two and three hour fever peaks [21].

### Pulmonary Illness Model.

Rabbits were administered CA-MRSA or purified Sag through intra-bronchial inoculation (2×109 colony-forming units [CFU] in 200 μl dialyzable beef heart medium, 100-200 μg purified SAg in 200 μl PBS, or 5 μg/kg SEB in PBS followed after 4 hr by 0.5 μg/kg LPS from Salmonella typhimurium in PBS [this latter model is referred to as “endotoxin enhancement” in which SAg pretreatment makes rabbits up to 106-fold more susceptible to LPS lethality]) [22]. Rabbits were anesthetized by subcutaneous injections of ketamine (25 mg/kg) and xylazine (25 mg/kg) (Phoenix Pharmaceuticals Inc., St. Joseph, Mo.) [23]. Once under anesthesia, their necks were shaved, and small incisions were made along the tracheas. Incisions (3 mm) were made in the tracheas, and polyethylene catheters (1 mm diameter, Fisher Scientific, Hampton, N.H.) were inserted and threaded into the left bronchi. Bacteria or purified SAgs were administered through the catheters. Once exposed to CA-MRSA or SAgs, rabbits were monitored for up to seven days for development of respiratory distress and lethal TSS (This point is defined as death, or in agreement with the University of Minnesota IACUC and 28 years research experience, failure to exhibit both escape behavior and ability to right themselves. At this point the animals were euthanized by intravenous [iv] administration of 1 ml/kg Beuthanasia D, [Schering-Plough Animal Health Corp., Union, N.J.]). Rabbits that did not develop respiratory distress and lethal TSS were euthanized after seven days.

### Immunizations.

Some Dutch belted rabbits were hyperimmunized against either purified TSST-1 or SEC prior to receiving intra-bronchial CA-MRSA or purified SAgs. SAgs (in phosphate buffered saline, PBS, 0.005M NaP04, pH 7.2, 0.15M NaCl) were mixed with equal volumes of incomplete Freund's adjuvant (Difco Laboratories, Detroit, Mich.). Final concentrations of 50 μg/ml of SAgs were used for injections, with each rabbit receiving 1.0 ml, injected subcutaneously into four sites on nape of the neck. Animals received initial injections, followed by booster injections every two weeks until antibody titers were >10,000 as determined by ELISA; antibody titers of >10,000 were considered hyperimmune. (Humans who do not develop menstrual TSS typically have IgG titers of 160.) For ELISA, wells of flat bottom 96-well plates were coated with 1.0 l-Ig/well purified TSST-1 or SEC [24] and washed. Rabbit sera were diluted serially 2-fold in the wells beginning with 1/10 dilutions, the plates were incubated for 2 hr at room temperature, and then wells were washed. Horseradish peroxidase-conjugated anti-rabbit IgG (Sigma-Aldrich, St. Louis, Mo.) were added to wells, the plates were incubated for 2 hr at room temperature, and the wells were again washed. The relative levels of IgG were determined by the addition color substrate containing 0-phenylenediamine and H20 2 (100 μl/well). Reactions were stopped by addition of 12.5% sulfuric acid (50 1-11), and then absorbances at 490 nm wavelength were measured spectophotometrically.

### Soluble High Affinity Vβ-T Cell Receptor (G5-8).

Some rabbits received soluble high-affinity VI3-TCR (100 ug iv daily) G5-8 in addition to CA-MRSA or SAgs. Soluble high affinity VI3-TCR G5-8 with specificity for SEB was generated by Vβ-TCR mutagenesis and selection by flow cytometry [25].

### Histology.

Rabbit lung tissue samples were fixed in 10% formalin and embedded in paraffin wax. Thick tissue sections (10 μm) were obtained using a microtome (Leica) RM2235, Wetzlar, Germany). Sections were stained with hematoxylin (Fisher Scientific, Fair Lawn, N.J.) and eosin (Sigma-Aldrich, St. Louis, Mo.) (H & E) following standard protocols at the University of Minnesota Veterinary Animal Pathology Laboratory.

### Statistical Analyses.

Data were analyzed by Fisher's Exact test. P values of ≦0.05 were considered significant. The Reed and Muench method was used to calculate the lethal dose 50% endpoint (LD50) for pulmonary exposure to purified SEC [26].

### Results

### CA-MRSA Rabbit Pulmonary Model.

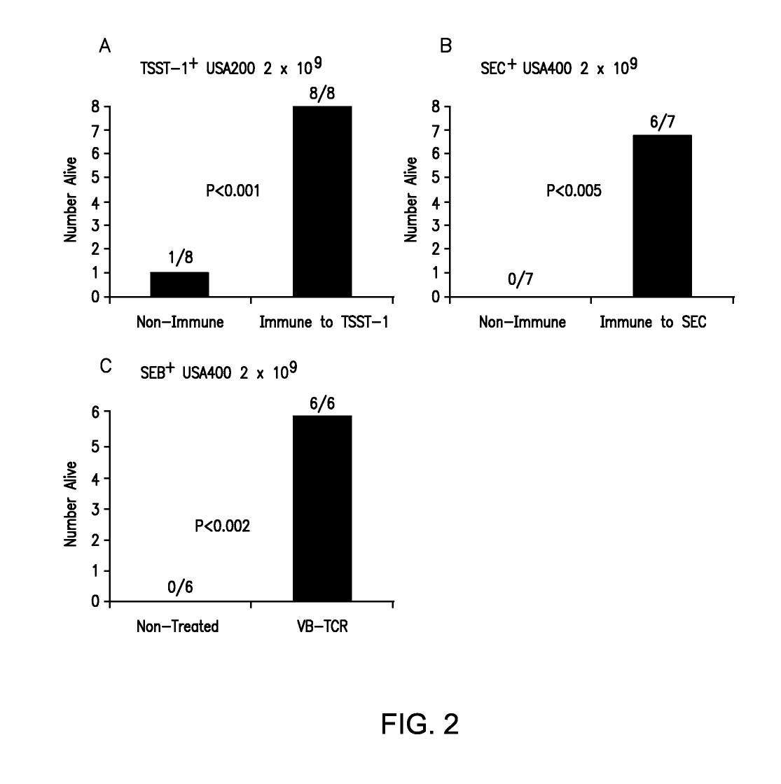

Rabbits were exposed intra-bronchial to CA-MRSA or purified SAgs, and monitored for signs of respiratory distress and lethal TSS. To initiate pulmonary infections, bacteria were administered into rabbit bronchi. CA-MRSA strains tested were USA200 MNPA, MN1021 or MN128 (TSST-1+; these strains do not produce the cytotoxins α-toxin+, β-toxin−, γ-toxin+, and PVL), and USA400 MW2 (SEC+, α-toxin+, β-toxin−, γ-toxin+, and PVL+) [4] and USA400 c99-529 (SEB+, α-toxin+, β-toxin−, γ-toxin+, and PVL+) [4]. Rabbits exposed to both USA200 and USA400 CA-MRSA organisms developed illness associated with respiratory distress and lethal TSS, and the animals succumbed (FIG. 2); only ⅛ animals exposed to the TSST-1+USA200 organisms survived (FIG. 2A), and no animals survived when challenged intra-bronchial with SEC+ USA400 MW2 organisms (FIG. 2 B).

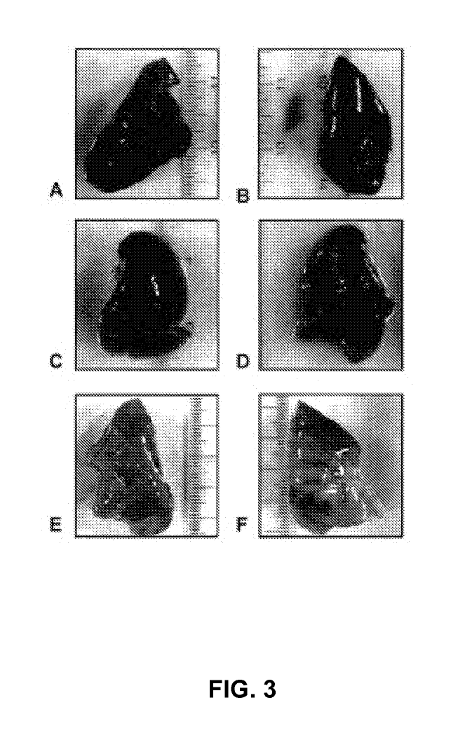

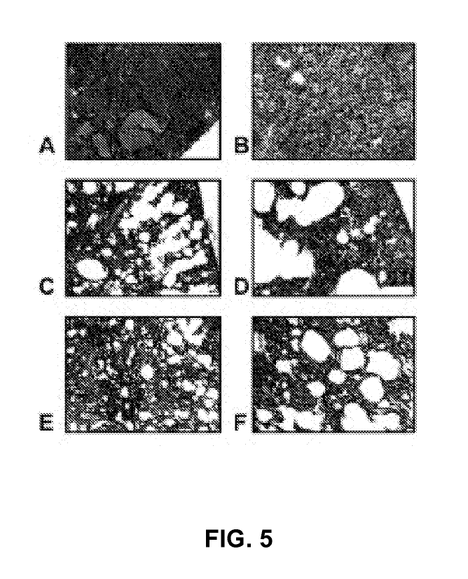

Excised lungs from rabbits exposed to CA-MRSA USA200 bacteria and USA400MW2 were severely hemorrhagic (FIGS. 3A-B and 4A-B), compared to rabbits challenged with PBS (FIGS. 3E-F and 4I-J). Histology of representative lung sections confirmed the presence of hemorrhagic tissue (FIGS. 5A-B and 6A-B), compared to rabbits challenged with PBS (FIGS. 5E-F and 61-J). Some groups of rabbits were hyperimmunized against highly purified TSST-1 or SEC (ELISA IgG titers >10,000) [14, 15] and then challenged intra-bronchially with corresponding CA-MRSA isolates that produce TSST-1 or SEC. Rabbits pre-immunized against TSST-1 or SEC did not develop respiratory distress or lethal TSS when challenged (p<0.001 and p<0.005, respectively) (FIG. 2A-B), and other than fever did not develop overt clinical signs.

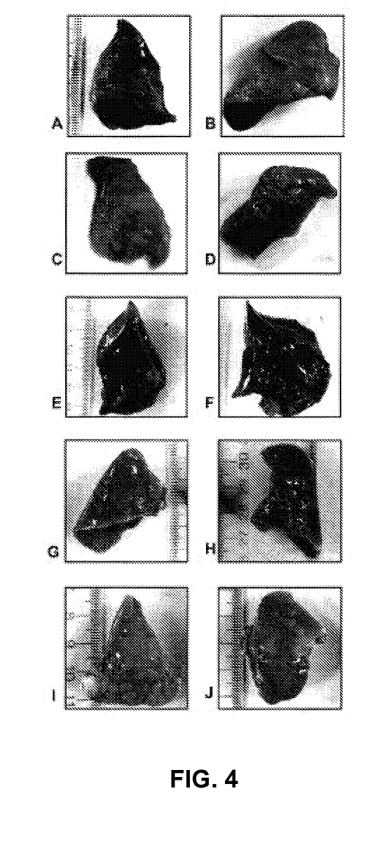

Excised lungs removed from non-immune animals challenged with CA-MRSA USA200 or USA400 MW2 showed hemorrhagic tissue (FIG. 3A-B and FIG. 4A-B). Lungs from TSST-1 or SEC hyperimmunized rabbits challenged with USA200 bacteria or USA400 MW2 did not have visible hemorrhagic lesions (FIG. 3C-D and FIG. 4C-D), though the lungs appeared somewhat congested, consistent with staphylococcal infections. Lungs from PBS-treated rabbits did not show hemorrhagic tissue and were not congested (FIG. 3E-F and FIG. 41-J).

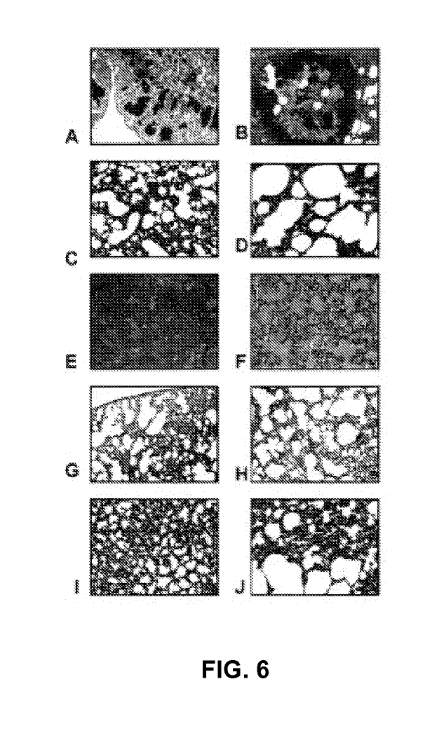

Histology showed hemorrhagic lung tissue in TSST-1 and SEC non-immune rabbits (FIG. 5A-B and FIG. 6A-B) challenged with CA-MRSA USA200 bacteria or MW2, and normal lung tissue in TSST-1 and SEC hyperimmunized rabbits challenged with USA200 and MW2 organisms (FIG. 5C-D and 6C-D) or PBS challenged rabbits (FIG. 5E-F and FIG. 61-J).

### Pulmonary Exposure to Purified SEC.

Rabbits were administered purified SEC (0 μg, 50 μg, 100 μg, or 200 μg) intra-bronchially in PBS. The LDso of SEC was 75 μg by this route. Post mortem examination of lungs from SEC-treated rabbits revealed hemorrhagic tissue (FIG. 4E-F), compared to normal tissues from PBS-treated animals (FIG. 41-J).

Rabbits were also hyperimmunized against purified SEC and then administered 200 μg SEC intra-bronchially in PBS. SEC hyperimmunized rabbits did not develop respiratory distress and lethal TSS (data not shown) over the seven day test period. In contrast, non-immunized rabbits exposed to 200 μg purified SEC showed severe respiratory distress and succumbed within 24 hr (data not shown). Excised lungs from non-immunized rabbits revealed the presence of hemorrhagic lesions (FIG. 4E-F). Lung samples from SEC hyperimmunized rabbits did not show hemorrhagic lesions (FIG. 4G-H); they resembled lungs from PBS-treated animals FIG. 41-J).

Histology confirmed that excised lung sections from non-immunized rabbits administered 200 μg SEC contained hemorrhagic lesions (FIG. 6E-F). Tissue from SEC hyperimmunized rabbits administered 200 μg SEC (FIG. 6G-H) and rabbits that received PBS (FIG. 61-J) showed normal lung tissue.

Passive Protection from SEB+ CA-MRSA or SEB by Vβ-TCR (G5-8).

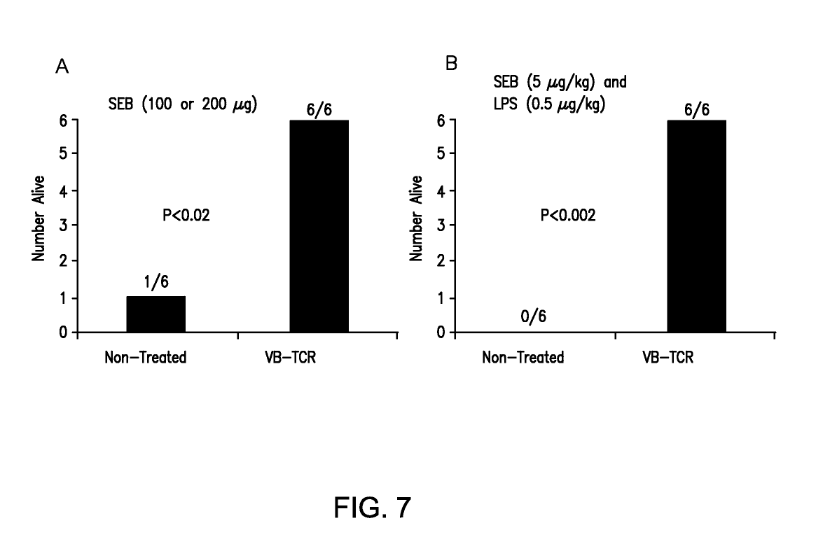

Additional experiments investigated the ability of soluble high affinity Vβ-TCR G5-8, specific for SEB [25], to provide passive protection from CA-MRSA or purified SEB intra-bronchial challenge. Rabbits that received high affinity Vβ-TCR G5-8 iv at the same time as the intra-bronchial SEB+ CA-MRSA c99-529 or purified SEB did not develop respiratory distress and lethal TSS (FIGS. 2C and 7A-B), whereas animals that did not receive G58 succumbed. Administration of high-affinity G5-8 2 hrs after intra-bronchial SEB also protected rabbits from respiratory distress and lethal TSS (FIG. 6B).

### Discussion

We evaluated the role of staphylococcal SAgs in serious pulmonary CA-MRSA infections and intoxications. Through TSST-1 and SEC immunization studies and use of soluble high-affinity Vβ-TCR G5-8 to neutralize SEB, we showed that these three SAgs, are critical for development of serious pulmonary TSS. We used rabbits as models since these animals are more similar to humans in susceptibility to SAgs [6, 27-31]; rabbits are also highly susceptible to cytotoxins [32]. Prior studies of CA-MRSA pulmonary infections used mice as the animal model [12, 33, 34] and have generated conflicting results regarding the roles of staphylococcal exotoxins, one group suggesting that PVL is critical to necrotizing pneumonia [34], while other groups suggesting a-toxin and phenol-soluble modulins, but not PVL, are critical [12, 33]. However, none of these studies assessed the role of SAgs in disease since mice are at least 10¹¹ more resistant to SAgs on a weight basis than humans [35, 36]. Indeed, the presence of SAgs increases the resistance of mice to infections (Sriskanden and Cohen). In contrast, young adult rabbits are only 10²-10³ more resistant than humans to SAgs, and rabbits ˜8 months of age are equally susceptible as humans to SAgs.

Prior hyperimmunization against TSST-1 protected rabbits from the lethality associated with intra-bronchial challenge with CA-MRSA USA200. Interestingly, these CA-MRSA strains do not produce a, β, γ, or PVL cytotoxins, yet cause fatal pulmonary TSS. The studies suggest that cytotoxins are not required for the fatal outcomes. Our hyperimmunization of rabbits against PVL followed by challenge with USA400 MW2 (SEC+, a-toxin+, β-toxin−, γ-toxin+, PVL+) resulted in lethal pulmonary illnesses, suggesting PVL is not critical for lethality (unpublished data). It appears that these redundantly expressed cytotoxins, including α-toxin, β-toxin, γ-toxin, and PVL, when produced, contribute to serious lung diseases, either through direct toxicity or induction of inflammation, but are not required for lethality in rabbits.

Presently, MRSA and their MSSA counterparts are highly significant causes of infectious disease deaths in the United States, including fatal pulmonary infections [3]. Our data suggest that SAgs are important contributors to those fatal infections. The initial report of CA-MRSA USA400 strains associated with deaths of four young children in the Upper Midwest demonstrated that 2 isolates produced SEB and the other 2 produced SEC [4]. A subsequent larger study of CA-MRSA USA400 strains indicated the vast majority produce either SEB or SEC [37]. It is also our experience that some regions of the United States are experiencing emergences of CA-MRSA USA100/200 S. aureus that produce TSST-1. As shown in the present studies, TSST-1 is critical in fatal pulmonary TSS associated with these strains.

Of great significance, our studies show that administration of soluble high-affinity Vβ-TCR G5-8 or prior hyperimmunization to raise neutralizing antibodies against SAgs dramatically increases rabbit survival. There are anecdotal studies of staphylococcal TSS patients being treated successfully with intravenous immunoglobulin (IVIG) [38, 39]. IVIG is highly capable of neutralizing SAgs [40]. Additionally, a study has shown that IVIG reduces the case:fatality rate of streptococcal TSS in humans [41]. However, IVIG is costly, requires large amounts of immunoglobulin to be administered, and has side effects. Our prior studies have shown that the soluble high-affinity Vβ-TCR G5-8 requires 2,200 times less than IVIG for comparable ability to neutralize SEB in rabbits. G5-8 is easy to prepare from bacterial clones, and requires only equimolar amounts to neutralize SEB, such small amounts for SEB neutralization that it may be possible to nebulize into the lungs, as well as administered iv.

### REFERENCES

1. Lowy F D. Staphylococcus aureus infections. N Engl J Med 1998; 339:520-32
2. Daum R S, Ito T, Hiramatsu K, et al. A novel methicillin-resistance cassette in community-acquired methicillin-resistant Staphylococcus aureus isolates of diversegenetic backgrounds. J Infect Dis 2002; 186:1344-7
3. Klevens R M, Morrison M A, Nadle J, et al. Invasive methicillin-resistant Staphylococcus aureus infections in the United States. Jama 2007; 298:1763-71
4. From the Centers for Disease Control and Prevention. Four pediatric deaths from community-acquired methicillin-resistant Staphylococcus aureus—Minnesota and North Dakota, 1997-1999. Jama 1999; 282:1123-5
5. Marrack P, Kappler J. The staphylococcal enterotoxins and their relatives. Science 1990; 248:705-11
6. McCormick J K, Yarwood J M and Schlievert P M. Toxic shock syndrome and bacterial superantigens: an update. Annu Rev Microbiol 2001; 55:77-104
7. Li H, L1era A, Malchiodi E L and Mariuzza R A. The structural basis of T cell activation by superantigens. Annu Rev Immunol 1999; 17:435-66
8. Wang L, Zhao Y, Li Z, et al. Crystal structure of a complete ternary complex of TCR, superantigen and peptide-MHC. Nat Struct Mol Bioi 2007; 14:169-71
9. Marrack P, Kappler J. The staphylococcal enterotoxins and their relatives. Science 1990; 248:1066
10. Schlievert P M. Staphylococcal enterotoxin B and toxic-shock syndrome toxin-1 are significantly associated with non-menstrual TSS. Lancet 1986; 1:1149-50
11. Schlievert P M, Tripp T J and Peterson M L. Reemergence of staphylococcal toxic shock syndrome in Minneapolis-St. Paul, Minn., during the 2000-2003 surveillance period. J Clin Microbiol 2004; 42:2875-6
12. Voyich J M, Otto M, Mathema B, et al. Is Panton-Valentine leukocidin the major virulence determinant in community-associated methicillin-resistant Staphylococcus aureus disease? J Infect Dis 2006; 194:1761-70
13. O'Reilly M, Kreiswirth B and Foster T J. Cryptic alpha-toxin gene in toxic shock syndrome and septicaemia strains of Staphylococcus aureus. Mol Microbiol 1990; 4:1947-55
14. Blomster-Hautamaa D A, Schlievert P M. Preparation of toxic shock syndrome toxin-1. Methods Enzymol 1988; 165:37-43
15. Blomster-Hautamaa D A, Kreiswirth B N, Novick R P and Schlievert P M. Resolution of highly purified toxic-shock syndrome toxin 1 into two distinct proteins by isoelectric focusing. Biochemistry 1986; 25:54-9
16. Schlievert P M, Shands K N, Dan B B, Schmid G P and Nishimura R D. Identification and characterization of an exotoxin from Staphylococcus aureus associated with toxic-shock syndrome. J Infect Dis 1981; 143:509-16
17. Bohach G A, Fast O J, Nelson R D and Schlievert P M. Staphylococcal and streptococcal pyrogenic toxins involved in toxic shock syndrome and related illnesses. Crit. Rev Microbiol 1990; 17:251-72
18. Laemmli U K. Cleavage of structural proteins during the assembly of the head of bacteriophage T4. Nature 1970; 227:680-5
19. Schlievert P M, Case L C, Nemeth K A, et al. Alpha and beta chains of hemoglobin inhibit production of Staphylococcus aureus exotoxins. Biochemistry 2007; 46:14349-58
20. Schlievert P M, Osterholm M T, Kelly J A and Nishimura R D. Toxin and enzyme characterization of Staphylococcus aureus isolates from patients with and without toxic shock syndrome. Ann Intern Med 1982; 96:937-40
21. Schlievert P M, Bettin K M and Watson O W. Effect of antipyretics on group A streptococcal pyrogenic exotoxin fever production and ability to enhance lethal endotoxin shock. Proc Soc Exp Bioi Med 1978; 157:472-5
22. Schlievert P M. Enhancement of host susceptibility to lethal endotoxin shock by staphylococcal pyrogenic exotoxin type C. Infect Immun 1982; 36:123-8
23. Schlievert P M, Gahr P J, Assimacopoulos A P, et al. Aggregation and binding substances enhance pathogenicity in rabbit models of Enterococcus faecalis endocarditis. Infect Immun 1998; 66:218-23
24. Hudson LaH, F. C. Practical Immunology. Blackwell Scientific Publications, 1980
25. Buonpane R A, Churchill H R, Moza B, et al. Neutralization of staphylococcal enterotoxin B by soluble, high-affinity receptor antagonists. Nat Med 2007; 13:725-9
26. Reed L J, Muench H. A simple method of estimating 50 percent endpoints. American Journal of Hygiene 1938; 27
27. Dinges M M, Orwin P M and Schlievert P M. Exotoxins of Staphylococcus aureus. Clin Microbiol Rev 2000; 13:16-34
28. Sriskandan S, Moyes 0, Buttery L K, et al. Streptococcal pyrogenic exotoxin A release, distribution, and role in a murine model of fasciitis and multiorgan failure due to Streptococcus pyogenes. J Infect Dis 1996; 173:1399-407
29. Giantonio B J, Alpaugh R K, Schultz J, et al. Superantigen-based immunotherapy: a phase I trial of PNU-214565, a monoclonal antibody-staphylococcal enterotoxin A recombinant fusion protein, in advanced pancreatic and colorectal cancer. J Clin Oncol 1997; 15:1994-2007
30. Dinges M M, Gregerson O S, Tripp T J, McCormick J K and Schlievert P M. Effects of total body irradiation and cyclosporin a on the lethality of toxic shock syndrome toxin-1 in a rabbit model of toxic shock syndrome. J Infect Dis 2003; 188:1142-5
31. Dinges M M, Schlievert P M. Comparative analysis of lipopolysaccharide-induced tumor necrosis factor alpha activity in serum and lethality in mice and rabbits pretreated with the staphylococcal superantigen toxic shock syndrome toxin 1. Infect Immun 2001; 69:7169-72
32. Dinges M M, Orwin P M and Schlievert P M. Exotoxins of Staphylococcus aureus. Clin Microbiol Rev 2000; 13:16-34
33. Bubeck Wardenburg J, Bae T, Otto M, Deleo F R and Schneewind 0. Poring over pores: alpha-hemolysin and Panton-Valentine leukocidin in Staphylococcus aureus pneumonia. Nat Med 2007; 13:1405-6 34. Labandeira-Rey M, Couzon F, Boisset S, et al. Staphylococcus aureus Panton-Valentine leukocidin causes necrotizing pneumonia. Science 2007; 315: 1130-3
35. Dinges M M, Jessurun J and Schlievert P M. Comparisons of mouse and rabbit models of toxic shock syndrome. International Congress and Symposium Series 1998; 229:167-168
36. Dinges M M, Schlievert P M. Comparative analysis of lipopolysaccharide-induced tumor necrosis factor alpha activity in serum and lethality in mice and rabbits pretreated with the staphylococcal superantigen toxic shock syndrome toxin 1. Infect Immun 2001; 69:7169-72.
37. Fey P O, Said-Salim B, Rupp M E, et al. Comparative molecular analysis of community- or hospital-acquired methicillin-resistant Staphylococcus aureus. Antimicrob Agents Chemother 2003; 47:196-203
38. Schlievert P M. Use of intravenous immunoglobulin in the treatment of staphylococcal and streptococcal toxic shock syndromes and related illnesses. J Allergy Clin Immunol 2001; 108:S107-10
39. Barry W, Hudgins L, Donta S T and Pesanti E L. Intravenous immunoglobulin therapy for toxic shock syndrome. JAMA 1992; 267:3315-6
40. Holm S E, Kohler W, Kaplan E L, et al. Streptococcal toxic shock syndrome (STSS). An update: a roundtable presentation. Adv Exp Med Bioi 1997; 418:193-9
41. Kaul R, McGeer A, Norrby-Teglund A, et al. Intravenous immunoglobulin therapy for streptococcal toxic shock syndrome—a comparative observational study. The Canadian Streptococcal Study Group. Clin Infect Dis 1999; 28:800-7

### Example 3

### Skin Hypersensitivity Reactions Caused by TSST-1 can be Prevented with the High-Affinity Vβ Neutralizing Drug D10

The Vβ neutralizing agent D10 was previously described in the paper: Buonpane, R. A., B. Moza, E. J. Sundberg, and D. M. Kranz (2005) Characterization of T cell receptors engineered for high affinity against toxic shock syndrome toxin-I. J. Mol. Biol. 353(2):308-321.

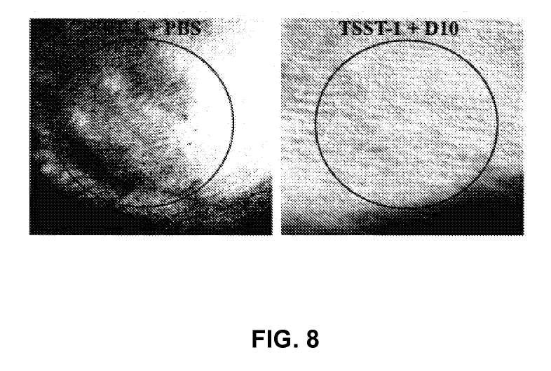

To examine if the D10 drug could be effective in skin diseases caused by, or exacerbated by, the bacterial exotoxins, a rabbit model was used. The experiments were used as provided in John, C. C., M. Niermann, B. Sharon, M. L. Peterson, D. M. Kranz, and P. M. Schlievert, 2009. Staphylococcal toxic shock syndrome erythroderma is associated with superantigenicity and hypersensitivity. Clin. Infect. Dis. 49:1893-6. For this experiment, the protein used was D10, a high-affinity Vβ protein for TSST-1 (rather than G5-8 for use with SEB). See FIG. 8 for these results.

### Example 4

### Wound Healing is Improved by Treatment with the Drug GS-8 when the Toxin SEB is Present

There has been evidence that wound healing is delayed by the presence of infectious agents, including Staphylococcus aureus. There is also evidence that the exotoxins secreted by S. aureus are at least in part responsible for this delayed wound healing, presumably due to their well-known inflammatory properties.

To examine whether a soluble high-affinity vβ directed against the toxin SEB could neutralize these inflammatory effects, an animal model of wound healing was examined. Animals were yellow or black, out-bred Dutch Belted Rabbits. An incision of 5 cm was made in each rabbit, and four sutures were applied at equal distances apart for each wound. Three groups of rabbis (3 per group) were used: 3 received PBS (phosphate buffered saline) at 1 ml volume/kg iv.; 3 received SEB in PBS at 10 μg/kg in 1 ml volume i.v. daily; and 3 received SEB in PBS at 10 μg/kg together with VβTCR G5-8 at 10 μg/kg in 1 ml volume of PBS i.v. daily.

The results shown in the photo in FIG. 9 were from day 10 of experimentation. All control and 2/3 G5-8 animals were healed by day 10; the other G5-8 animal healed by day 14. The SEB treated animals healed by day 21. All treatments were stopped on day 10 to give SEB animals the ability to heal. Thus, the treatment group results were designated as healed or unhealed as follows:
PBS: one yellow, two black, 3/3 healed
SEB: two yellow, one black, 0/3 healed
SEB and G5-8: one yellow, two black, 2/3 healed, 1/3 partially healed

The presence of the SEB-neutralizing agent is able to allow wound healing to proceed normally, without the delay caused by the toxin SEB. Further, Vβ proteins with high affinity for the bacterial toxins are effective in the treatment of wounds suspected of having infections due to S. aureus. These results are discussed in FIG. 9 and elsewhere herein.

### Example 5

### In Vitro and in Vivo Inhibition of SEC and TSST-1 Using High Affinity Vbeta Proteins L3 and D10V

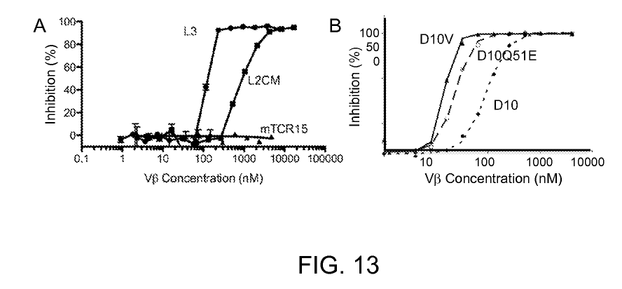

FIG. 13 shows in vitro inhibition of SEC and TSST-1 using high affinity Vbeta proteins L3 and D10V. In the experiments, different concentrations of the Vbeta drugs were used to show inhibition activity against SEC and TSST-1.

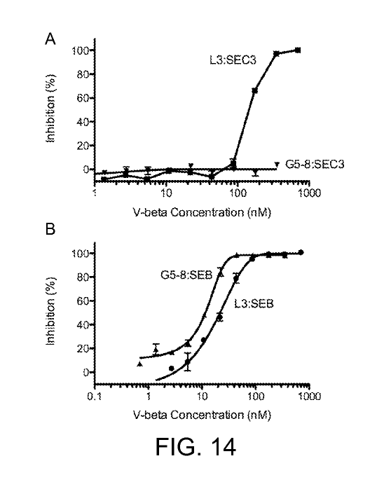

FIG. 14 shows in vitro inhibition of SEC and SEB using high affinity Vbeta proteins L3 and G5-8. In the experiments, different concentrations of the Vbeta drugs were used to show inhibition activity against SEC and TSST-1.

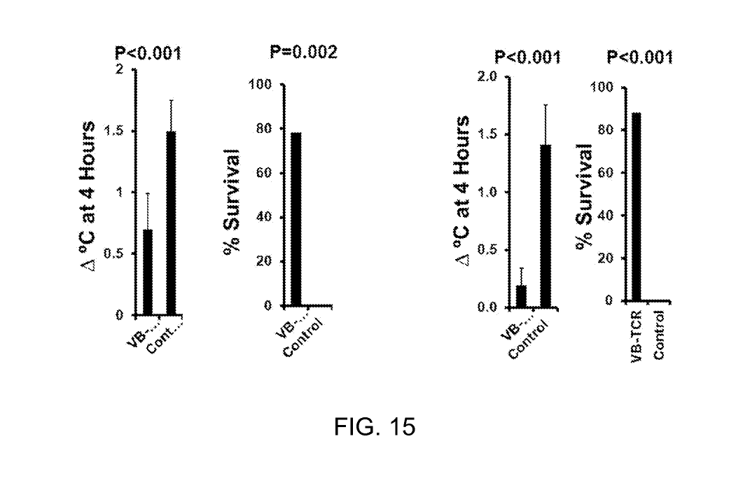

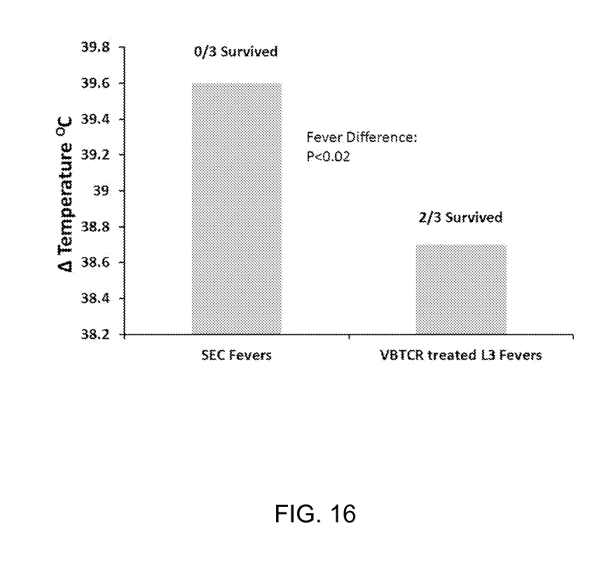

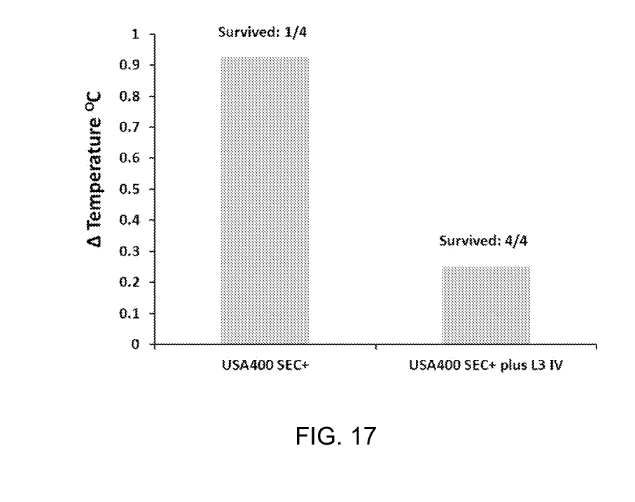

FIGS. 15-18 show in vivo inhibition of SEC+, SEC and TSST-1 with V beta drugs D10V or L3 in rabbits. The experiments were performed using the protocols described above. The results show the V beta drugs improve survival as compared to the control experiments with PBS and decreased the fever associated with SEC exposure.

### INCORPORATION BY REFERENCE

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

PCT publication WO2007/106894 (PCT/US2007/064085) and information therein, including the sequences is specifically incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, synthetic methods, and compositions other than those specifically exemplified can be employed in the practice of the invention without resort to undue experimentation. All art-known functional equivalents, of any such methods, device elements, starting materials, synthetic methods, and compositions are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the invention.

All patents and publications mentioned in the specification are indicative of the levels of skill of those skilled in the art to which the invention pertains.

One skilled in the art readily appreciates that the present invention is well adapted to carry out the objects and obtain the ends and advantages mentioned, as well as those inherent in the present invention. The methods, components, materials and dimensions described herein as currently representative of preferred embodiments are provided as examples and are not intended as limitations on the scope of the invention. Changes therein and other uses which are encompassed within the spirit of the invention will occur to those skilled in the art, are included within the scope of the claims.

Although the description herein contains certain specific information and examples, these should not be construed as limiting the scope of the invention, but as merely providing illustrations of some of the embodiments of the invention. Thus, additional embodiments are within the scope of the invention and within the following claims.

### Administration and Formulation

### Salts and Prodrugs

Compounds of the invention can have prodrug forms. Prodrugs of the compounds of the invention are useful in embodiments including compositions and methods. Any compound that will be converted in vivo to provide a biologically, pharmaceutically, diagnostically, or therapeutically active form of a compound of the invention is a prodrug. Various examples and forms of prodrugs are well known in the art. Examples of prodrugs are found, inter alia, in: Design of Prodrugs, edited by H. Bundgaard, (Elsevier, 1985); Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191 (1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392). A prodrug, such as a pharmaceutically acceptable prodrug, can represent prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of a compound described herein, for example, by hydrolysis in blood or by other cell, tissue, organ, or system processes. Further discussion is provided in: T. Higuchi and V. Stella, Pro-drugs as Novel Delivery Systems, V. 14 of the A.C.S. Symposium Series; and in Edward B. Roche, ed., Bioreversible Carriers in Drug Design, American Pharmaceutical Association and Pergamon Press (1987).

Compounds of the invention can be formulated with pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include, among others, halides (e.g., F⁻, Cl⁻, Br⁻, At⁻), sulfate, acetates (e.g., acetate, trifluoroacetate), ascorbates, aspartates, benzoates, citrates, and lactate.

Pharmaceutically acceptable salts comprise pharmaceutically-acceptable anions and/or cations. As used herein, the term “pharmaceutically acceptable salt” can refer to acid addition salts or base addition salts of the compounds in the present disclosure. A pharmaceutically acceptable salt is any salt which retains at least a portion of the activity of the parent compound and does not impart significant deleterious or undesirable effect on a subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include, but are not limited to, acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts can be derived from amino acids, including, but not limited to, cysteine. Other pharmaceutically acceptable salts can be found, for example, in Stahl et al., Handbook of Pharmaceutical Salts Properties, Selection, and Use, Wiley-VCH, Verlag Helvetica Chimica Acta, Zurich, 2002. (ISBN 3-906390-26-8).

### Efficacy

Typically, a compound of the invention, or pharmaceutically acceptable salt thereof, is administered to a subject in a diagnostically or therapeutically effective amount. One skilled in the art generally can determine an appropriate dosage.

Compositions for oral administration can be, for example, prepared in a manner such that a single dose in one or more oral preparations contains at least about 20 mg of the compound per square meter of subject body surface area, or at least about 50, 100, 150, 200, 300, 400, or 500 mg of the compound per square meter of subject body surface area (the average body surface area for a human is, for example, 1.8 square meters). In particular, a single dose of a composition for oral administration can contain from about 20 to about 600 mg, and in certain aspects from about 20 to about 400 mg, in another aspect from about 20 to about 300 mg, and in yet another aspect from about 20 to about 200 mg of the compound per square meter of subject body surface area. Compositions for parenteral administration can be prepared in a manner such that a single dose contains at least about 20 mg of the compound per square meter of subject body surface area, or at least about 40, 50, 100, 150, 200, 300, 400, or 500 mg of the compound per square meter of subject body surface area. In particular, a single dose in one or more parenteral preparations contains from about 20 to about 500 mg, and in certain aspects from about 20 to about 400 mg, and in another aspect from about 20 to about 450 mg, and in yet another aspect from about 20 to about 350 mg of the compound per square meter of subject body surface area. It should be recognized that these oral and parenteral dosage ranges represent generally preferred dosage ranges, and are not intended to limit the invention. The dosage regimen actually employed can vary widely, and, therefore, can deviate from the generally preferred dosage regimen. It is contemplated that one skilled in the art will tailor these ranges to the individual subject.

Toxicity and therapeutic efficacy of such compounds and bioconjugates can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compounds and bioconjugates that exhibit large therapeutic indices are preferred. While compounds and bioconjugates exhibiting toxic side effects can be used, care should be taken to design a delivery system that targets such compounds and bioconjugates to the site affected by the disease or disorder in order to minimize potential damage to unaffected cells and reduce side effects.

Data obtained from the cell culture assays and animal studies can be used in formulating a range of dosages for use in humans and other mammals. The dosage of such compounds and bioconjugates lies preferably within a range of circulating plasma or other bodily fluid concentrations that include the ED₅₀ and provides clinically efficacious results (i.e., reduction in disease symptoms). The dosage can vary within this range depending upon the dosage form employed and the route of administration utilized. For any compound and bioconjugate of the present invention, the therapeutically effective amount can be estimated initially from cell culture assays. A dosage can be formulated in animal models to achieve a circulating plasma concentration range that includes the ED₅₀ (the concentration of the test compound that achieves a half-maximal inhibition of symptoms) as determined in cell culture. Such information can be used to more accurately determine useful dosages in humans and other mammals. Compound and bioconjugate levels in plasma can be measured, for example, by high performance liquid chromatography.

An amount of a compound or bioconjugate that can be combined with a pharmaceutically acceptable carrier to produce a single dosage form will vary depending upon the patient treated and the particular mode of administration. It will be appreciated by those skilled in the art that the unit content of a compound/bioconjugate contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage and dosage regime for treating a disease or condition can be selected in accordance with a variety of factors, including the type, age, weight, sex, diet and/or medical condition of the patient, the route of administration, pharmacological considerations such as activity, efficacy, pharmacokinetic and/or toxicology profiles of the particular compound/bioconjugate employed, whether a compound/bioconjugate delivery system is utilized, and/or whether the compound/bioconjugate is administered as a pro-drug or part of a drug combination. Thus, the dosage regime actually employed can vary widely from subject to subject, or disease to disease and different routes of administration can be employed in different clinical settings.

The identified compounds/bioconjugates monitor, treat, inhibit, control and/or prevent, or at least partially arrest or partially prevent, diseases and conditions of interest and can be administered to a subject at therapeutically effective amounts and optionally diagnostically effective amounts. Compositions/formulations of the present invention comprise a therapeutically effective amount (which can optionally include a diagnostically effective amount) of at least one compound or bioconjugate of the present invention. Subjects receiving treatment that includes a compound/bioconjugate of the invention are preferably animals (e.g., mammals, reptiles and/or avians), more preferably humans, horses, cows, dogs, cats, sheep, pigs, and/or chickens, and most preferably humans.

### Administration

The preferred composition depends on the route of administration. Any route of administration can be used as long as the target of the compound or pharmaceutically acceptable salt is available via that route. Suitable routes of administration include, for example, oral, intravenous, parenteral, inhalation, rectal, nasal, topical (e.g., transdermal and intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual, and intestinal administration.

In an embodiment, the invention provides a method for treating a medical condition comprising administering to a subject (e.g. patient) in need thereof, a therapeutically effective amount of a composition of the invention. In an embodiment, the invention provides a method for diagnosing or aiding in the diagnosis of a medical condition comprising administering to a subject in need thereof, a diagnostically effective amount of a composition of the invention. In an embodiment, the medical condition is cancer, or various other diseases, injuries, and disorders, including cardiovascular disorders such as atherosclerosis and vascular restenosis, inflammatory diseases, ophthalmic diseases and dermatological diseases. When used herein, the terms “diagnosis”, “diagnostic” and other root word derivatives are as understood in the art and are further intended to include a general monitoring, characterizing and/or identifying a state of health or disease. The term is meant to encompass the concept of prognosis. For example, the diagnosis of pneumonia can include an initial determination and/or one or more subsequent assessments regardless of the outcome of a previous finding. The term does not necessarily imply a defined level of certainty regarding the prediction of a particular status or outcome.

The diagnostic and therapeutic formulations of this invention can be administered alone, but can be administered with a pharmaceutical carrier selected upon the basis of the chosen route of administration and standard pharmaceutical practice.

Any suitable form of administration can be employed in connection with the diagnostic and therapeutic formulations of the invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations can also include aerosol formulation, creams, gels, solutions, etc. The present compositions, preparations and formulations are administered in doses effective to achieve the desired diagnostic and/or therapeutic effect. Such doses can vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations can also optionally include stabilizing agents and skin penetration enhancing agents.

The exact formulation, route of administration and dosage can be chosen by the individual physician in view of the patient's condition (see e.g. Fingl et. al., in The Pharmacological Basis of Therapeutics, 1975, Ch. 1 p. 1).

It should be noted that the attending physician would know how to and when to terminate, interrupt, or adjust administration due to toxicity, or to organ dysfunctions, or other side effects. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

1-44. (canceled)

45. A method for preventing or reducing the toxic effects of a bacterial superantigen in a subject comprising, administering to the subject an effective amount of a neutralizing agent that binds to the bacterial superantigen, wherein the neutralizing agent prevents or reduces binding of the bacterial superantigen to a T cell receptor variable region or prevents or reduces crosslinking of the T cell receptor variable region and an MHC molecule; wherein the neutralizing agent binds to the bacterial superantigen with an equilibrium constant KD of between about 10⁻⁸M and 10⁻¹²M; thereby preventing or reducing the toxic effects of the bacterial superantigen.

46. The method of claim 45 wherein the neutralizing agent binds to the bacterial superantigen with an equilibrium constant KD of about 48 pM.

47. The method of claim 45 wherein the neutralizing agent has increased in vivo activity as compared to an equivalent amount of IVIG.

48. The method of claim 45 wherein the neutralizing agent has an in vivo activity about 2,200 fold higher than an IVIG.

49. The method of claim 45 wherein the neutralizing agent is a V beta fragment.

50. The method of claim 49 wherein the V beta fragment is selected from the group consisting of SEQ ID NOs:16-22, 30-44 and 66-73.

51. The method of claim 45 wherein the bacterial superantigen is selected from the group consisting of TSST-1, SEB and SEC.

52. The method of claim 45 wherein the toxic effects of the bacterial superantigen are associated with delayed wound healing, toxic shock syndrome (TSS), pneumonia, pulmonary TSS, necrotizing pneumonia, extreme pyrexia, or atopic dermatitis.

53. A method for treating Staphylococcus aureus infection in a subject comprising, administering to the subject an effective amount of a neutralizing agent that binds to a bacterial superantigen produced by the Staphylococcus aureus, wherein the neutralizing agent prevents or reduces binding of the bacterial superantigen to a T cell receptor variable region or prevents or reduces crosslinking of the T cell receptor variable region and an MHC molecule; wherein the neutralizing agent binds to the bacterial superantigen with an equilibrium constant KD of between about 10⁻⁸M and 10⁻¹²M; thereby treating the Staphylococcus aureus infection.

54. The method of claim 53 wherein the neutralizing agent binds to the bacterial superantigen with an equilibrium constant KD of about 48 pM.

55. The method of claim 53 wherein the neutralizing agent has increased in vivo activity as compared to an equivalent amount of IVIG.

56. The method of claim 53 wherein the neutralizing agent has an in vivo activity about 2,200 fold higher than an IVIG.

57. The method of claim 53 wherein the neutralizing agent is a V beta fragment.

58. The method of claim 57 wherein the V beta fragment is selected from the group consisting of SEQ ID NOs:16-22, 30-44 and 66-73.

59. The method of claim 53 wherein the bacterial superantigen is selected from the group consisting of TSST-1, SEB and SEC.

60. The method of claim 53 wherein the toxic effects of the bacterial superantigen are associated with delayed wound healing, toxic shock syndrome (TSS), pneumonia, pulmonary TSS, necrotizing pneumonia, extreme pyrexia, or atopic dermatitis.

61. A bacterial superantigen neutralizing agent that is effective in a subject, comprising a protein, or fragment thereof, which binds to the bacterial superantigen with an equilibrium constant KD between about 10⁻⁸M and 10⁻¹²M.

62. The bacterial superantigen neutralizing agent of claim 61 wherein the agent binds to the bacterial superantigen with an equilibrium constant KD of at least 48 pM.

63. The bacterial superantigen neutralizing agent of claim 61 wherein an amount of the protein has increased in vivo activity as compared to an equivalent amount of IVIG.

64. The bacterial superantigen neutralizing agent of claim 61 wherein the neutralizing agent has an in vivo activity about 2,200 fold higher than an IVIG.

65. The bacterial superantigen neutralizing agent of claim 61 wherein the neutralizing agent is a V beta fragment.

66. The bacterial superantigen V beta fragment of claim 65 wherein the V beta fragment is selected from the group consisting of SEQ ID NOs:16-22, 30-44 and 66-73.

67. The bacterial superantigen neutralizing agent of claim 65 wherein the bacterial superantigen is selected from the group consisting of TSST-1, SEB and SEC.

68. The bacterial superantigen neutralizing agent of claim 65 wherein the toxic effects of the bacterial superantigen are associated with delayed wound healing, toxic shock syndrome (TSS), pneumonia, pulmonary TSS, necrotizing pneumonia, extreme pyrexia, or atopic dermatitis.


SEQUENCE LISTING (79 sequences)

SEQ ID NO: 1 - protein, 118 aa, Homo sapiens
GAVVSQHPSRVIAKSGTSVKIECRSLDFQATTMFWYRQFPKQSLMLMATSNEGSKATYEQGVEKDKFLINHASLTLSTLTVTSAHPEDSSFYICSALAGSGSSTDTQYFGPGTRLTVL

SEQ ID NO: 2 - protein, 118 aa, Homo sapiens
GAVVSQHPSRVIAKSGTSVKIECRPLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 3 - protein, 118 aa, Homo sapiens
GAVVSQHPSRVIAKSGTSVKIECRTLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 4 - protein, 118 aa, Homo sapiens
GAVVSQHPSMVIAKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 5 - protein, 118 aa, Homo sapiens
GAVVSQHPSRVIVKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTRLTVL

SEQ ID NO: 6 - protein, 118 aa, Homo sapiens
GAVVSQHPSRVIAKSGTSVKIECRSLDFQATTMFWYRQFPKQSLMLMATSNEGSKATYEQGVEKDKFLINHASLTLSTLTVTSAHPEDSGFYICSALAGSGSSTDTQYFGPGTQLTVL

SEQ ID NO: 7 - protein, 118 aa, Homo sapiens
GAVVSQHPSRVIAKSGTSVKIECRSLDFQATTMFWYRQFPKQSLMLMATSNVGSKATYEQGVEKDKFLINHASPTLSTLTVTSAHPEDSSFYICSALAGSGSSTDTQYFGPGTRLTVL

SEQ ID NO: 8 - protein, 118 aa, Homo sapiens
GAVVSQHPSRVIAKSGTSVKIECRSLDFQATTMFWYRQFPKQSLMLMATSNEGSKATYEQGVEKDKFLINHASPTLSTLTVTSAHPEDSGFYVCSALAGSGSSTDTQYFGPGTRLTVL

SEQ ID NO: 9 - protein, 118 aa, Homo sapiens
GAVVSQHPSRVIAKSGTSVKIECRSLDFQATTMFWYRQFPKQSLMLMATSHLDMHATYEQGVEKDKFLINHASPTLSTLTVTSAHPEDSGFYICSALAGSGSSTDTQYFGPGTQLTVL

SEQ ID NO: 10 - protein, 118 aa, Homo sapiens
GAVVSQHPSMVIVKSGTSVKIECRSLDFQATTMFWYRQFPKQSLMLMATSRIDFHATYEQGVEKDKFLINHASPTLSTLTVTSAHPEDSGFYICSALAGSGSSTDTQYFGPGTQLTVL

SEQ ID NO: 11 - protein, 118 aa, Homo sapiens
GAVVSQHPSMVIAKSGTSVKIECRSLDFQATTMFWYRQFPKQSLMLMATSRMDYHATYEQGVEKDKFLINHASPTLSTLTVTSAHPEDSGFYICSALAGSGSSTDTQYFGPGTRLTVL

SEQ ID NO: 12 - protein, 118 aa, Homo sapiens
GAVVSQHPSRVIVKSGTSVKIECRSLDFQATTMFWYRQFPKQSLMLMATSRLWDSATYEQGVVKDKFLINHASPTLSTLTVTSAHPEDSGFYICSALAGSGSSTDTQYFGPGTQLTVL

SEQ ID NO: 13 - protein, 118 aa, Homo sapiens
GAVVSQHPSRVIVKSGTSVKIECRTLDFQATTMFWYRQFPKQSLMLMATSHQGFNAIYEQGVEKDKFLINHASPTLSTLTVTSAHPEDSGFYVCSALAGSGSSTDTQYFGPGTRLTVL

SEQ ID NO: 14 - protein, 118 aa, Homo sapiens
GAVVSQHPSRVIVKSGTSVKIECRSLDFQATTMFWYRQFPKQSLMLMATSRFGYHATYEQGVEKDKFLINHASPTLSTLTVTSAHPEDSGFYVCSALAGSGSSTDTQYFGPGTRLTVL

SEQ ID NO: 15 - protein, 118 aa, Homo sapiens
GAVVSQHPSRVIVKSGTSVKIECRSLDFQATTMFWYRQFPKQSLMLMATSRPGFHATYEQGVEKDKFLINHASPTLSTLTVTSAHPEDSGFYVCSALAGSGSSTDTQYFGPGTRLTVL

SEQ ID NO: 16 - protein, 118 aa, Homo sapiens
GAVVSQHPSRVIVKSGTSVKIECRSLERGSATMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 17 - protein, 118 aa, Homo sapiens
GAVVSQHPSMVIVKSGTSVKIECRSLNFFSGTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 18 - protein, 118 aa, Homo sapiens
GAVVSQHPSMVIVKSGTSVKIECRSLDFQATTMFWYRQFPKQSLMLMATSRIDFHATYEQGVVKDKFLINHASPTLSTLTVTSAHPEDSGFYVCSALAGSGSSTDTQYFGPGTQLTVL

SEQ ID NO: 19 - protein, 118 aa, Homo sapiens
GAVVSQHPSMVIVKSGTSVKIECRSLGTPFDTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 20 - protein, 118 aa, Homo sapiens
GAVVSQHPSMVIVKSGTSVKIECRSLDTNIHTMFWYRQFPKQSLMLMATSHQGFNAIYEQGVVKDKFLINHASPTLSTLTVTSAHPEDSGFYVCSALAGSGSSTDTQYFGPGTQLTVL

SEQ ID NO: 21 - protein, 118 aa, Homo sapiens
GAVVSQHPSMVIVKSGTSVKIECRSLDGFSGTMFWYRQFPKQSLMLMATSHQGFNAIYEQGVVKDKFLINHASPTLSTLTVTSAHPEDSGFYICSALAGSGSSTDTQYFGPGTQLTVL

SEQ ID NO: 22 - protein, 118 aa, Homo sapiens
GAVVSQHPSRVIVKSGTSVKIECRSLNVAGSTMFWYRQFPKQSLMLMATSHQGFNAIYEQGVVKDKFLINHASPTLSTLTVTSAHPEDSGFYICSALAGSGSSTDTQYFGPGTRLTVL

SEQ ID NO: 23 - protein, 110 aa, Mus musculus
EAVVTQSPRNKVAVTGGKVTLSCNQTNNHNNMYWYRQDTGHGLRLIHYSYGAGSTEKGDIPDGYKASRPSQENFSLILELATPSQTSVYFCASGGGGTLYFGAGTRLSVL

SEQ ID NO: 24 - protein, 110 aa, Mus musculus
EAVVTQSPRNKVAVTGEKVTLSCNQTNNHNNMYWYRQDTGHGLRLIHYSYGAGSTEKGDIPDGYKASRPSQENFSLILELATPSQTSVYFCASGGGGTLYFGAGTRLSVL

SEQ ID NO: 25 - protein, 110 aa, Mus musculus
EAVVTQSPRNKVAVTGEKVTLSCNQTNNHNNMYWYRQDTGHGLRLIHYSYGVGNTEKGDIPDGYEASRPSHENFSLILVSATPSQSSVYFCASGVGGTLYFGAGTRLSVL

SEQ ID NO: 26 - protein, 110 aa, Mus musculus
EAVVTQSPRNKVAVTGEKVTLSCNQTNNHNNMYWYRQDTGHGLRLIHYSLGIHSTEKGDIPDGYKASRPSQENFSLILELATPSQTSVYFCASGGGGTLYFGAGTRLSVL

SEQ ID NO: 27 - protein, 110 aa, Mus musculus
EAVVTQSPRNKVAVTGEKVTLSCNQTNNHNNMYWYRQDTGHELRLIHYSLGIHNVRRGDIPDGYKASRPSQENFSLILELATPSQTSVYFCASGGGGTLYFGAGTRLSVL

SEQ ID NO: 28 - protein, 110 aa, Mus musculus
EAVVTQSPRNKVAVTGEKVTLSCNQTNNHNNMYWYRQDTGHELRLIFMSHGIHNVEKGDIPDGYKASRPSQENFSLILELATPSQTSVYFCASGGGGTLYFGAGTRLSVL

SEQ ID NO: 29 - protein, 110 aa, Mus musculus
EAVVTQSPRNKVAVTGEKVTLSCKQTNNHNNMYWYRQDTGHELRLIFMSHGIRNVEKGDIPDGYKASRPSQENFSLILELATPSQTSVYFCASGGGGTLYFGAGTRLSVL

SEQ ID NO: 30 - protein, 111 aa, Mus musculus
EAVVTQSPRNKVAVTGEKVTLSCKQTNTYLNNMYWYRQDTGHELRLIFMSHGIRNVEKGDIPDGYKASRPSQENFSLILELATPSQTSVYFCASGGGGTLYFGAGTRLSVL

SEQ ID NO: 31 - protein, 111 aa, Mus musculus
EAVVTQSPRNKVAVTGEKVTLSCKQTCSYLDNMYWYRQDTGHELRLIFMSHGIRNVEKGDIPDGYKASRPSQENFSLILELATPSQTSVYFCASGGGGTLYLGAGTRLSVL

SEQ ID NO: 32 - protein, 111 aa, Mus musculus
EAVVTQSPRNKVAVTGEKVTLSCKQTGSYFDNMYWYRQDTGHELRLIFMSHGIRNVEKGDIPDGYKASRPSQENFSLILELATPSQTSVYFCASGGGGTLYFGAGTRLSVL

SEQ ID NO: 33 - protein, 111 aa, Mus musculus
EAVVTQSPRNKVAVTGEKVTLSCKQTKGYNDNMYWYRQDTGHELRLIFMSHGIRNVEKGDIPDGYKASRPSQENFSLILELATPSQTSVYFCASGGGGTLYFGAGTRLSVL

SEQ ID NO: 34 - protein, 111 aa, Mus musculus
EAVVTQSPRNKVAVTGEKVTLSCKQTNTYWNNMYWYRQDTGHELRLIFMSHGIRNVEKGDIPDGYKASRPSQENFSLILELATPSQTSVYFCASGGGGTLYFGAGTRLSVL

SEQ ID NO: 35 - protein, 111 aa, Mus musculus
EAVVTQSPRNKVAVTGEKVTLSCKQTNSYFDNMYWYRQDTGHELRLIFMSHGIRNVEKGDIPDGYKASRPSQENFSLILELATPSQTSVYFCASGGGGTLYFGAGTRLSVL

SEQ ID NO: 36 - protein, 111 aa, Mus musculus
EAVVTQSPRNKVAVTGEKVTLSCKQTNSYFNNMYWYRQDTGHELRLIFMSHGIRNVEKGDIPDGYKASRPSQENFSLILELATPSQTSVYFCASGGGGTLYFGAGTRLSVL

SEQ ID NO: 37 - protein, 111 aa, Mus musculus; identical to SEQ ID NO: 36

SEQ ID NO: 38 - protein, 110 aa, Mus musculus
EAVVTQSPRNKVAVTGEKVTLSCKQRDRWNNMYWYRQDTGHELRLIFMSHGIRNVEKGDIPDGYKASRPSQENFSLILELATPSQTSVYFCASGGGGTLYFGAGTRLSVL

SEQ ID NO: 39 - protein, 111 aa, Mus musculus
EAVVTQSPRNKVAVTGEKVTLSCKQTNSYLDNMYWYRQDTGHELRLIFMSHGIRNVEKGDIPDGYKASRPSQENFSLILELATPSQTSVYFCASGGGGTLYFGAGTRLSVL

SEQ ID NO: 40 - protein, 110 aa, Mus musculus; identical to SEQ ID NO: 38

SEQ ID NO: 41 - protein, 110 aa, Mus musculus
EAVVTQSPRNKVAVTGEKVTLSCKQTNNHNNMYWYRQDPGHELRLIFMSHGIRNVEKGDIPDGYKASRPSQENFPLILELATPSQTSVYFCASGGGGTLYFGAGTRLSVL

SEQ ID NO: 42 - protein, 111 aa, Mus musculus
EAVVTQSPRNKVAVTGEKVTLSCKQTNSYFPNMYWYRQDTGHELRLIFMSHGIRNVEKGDIPDGYKASRPSQENFSLILELATPSQTSVYFCASGGGGTLYFGAGTRLSVL

SEQ ID NO: 43 - protein, 111 aa, Mus musculus
EAVVTQSPRNKVAVTGEKVTLSCKQTIGYLNNMYWYRQDTGHELRLIFMSHGIRNVEKGDIPDGYKASRPSQENFSLILELATPSQTSVYFCASGGGGTLYFGAGTRLSVL

SEQ ID NO: 44 - protein, 110 aa, Mus musculus; identical to SEQ ID NO: 38

SEQ ID NO: 45 - protein, 110 aa, Mus musculus; identical to SEQ ID NO: 23

SEQ ID NO: 46 - protein, 110 aa, Mus musculus; identical to SEQ ID NO: 24

SEQ ID NO: 47 - protein, 110 aa, Mus musculus
EAVVTQSPRNKVAVTGEKVTLSCNQTNNHNNMYWYRQDTGHELRLIHYSLGIHSTEKGDIPDGYKASRPSQENFSLILELATPSQTSVYFCASGGGGTLYFGAGTRLSVL

SEQ ID NO: 48 - protein, 110 aa, Mus musculus
EAVVTQSPRNKVAVTGEKVTLSCNQTNNHNNMYWYRQDTGHELRLIHYSLGIRSTEKGDIPDGYKASRPSQENFSLILELATPSQTSVYFCASGGGGTLYFGAGTRLSVL

SEQ ID NO: 49 - protein, 110 aa, Mus musculus
EAVVTQSPRNKVAVTGEKVTLSCNQTNNHNNMYWYRQDTGHGLRLIHYSNGVRSTEKGDIPDGYKASRPSQENFSLILELATPSQTSVYFCASGGGGTLYFGAGTRLSVL

SEQ ID NO: 50 - protein, 110 aa, Mus musculus
EAVVTQSPRNKVAVTGEKVTLSCNQTNNHNNMYWYRQDTGHGLRLIHYSYGAGHIGKGDIPDGYKASRPSQENFSLILELATPSQTSVYFCASGGGGTLYFGAGTRLSVL

SEQ ID NO: 51 - protein, 110 aa, Mus musculus
EAVVTQSPRNKVAVTGEKVTLSCNQTNNHNNMYWYRQDTGHGLRLIYYSYGAGHLLSGDIPDGYKASRPSQENFSLILELATPSQTSVYFCASGGGGTLYFGAGTRLSVL

SEQ ID NO: 52 - protein, 110 aa, Mus musculus
EAVVTQSPRNKVAVTGEKVTLSCNQTNNHNNMYWYRQDTGHGLRLIYYSYGAGHVGKGDIPDGYKASRPSQENFSLILELATPSQTSVYFCASGGGGTLYFGAGTRLSVL

SEQ ID NO: 53 - protein, 110 aa, Mus musculus
EAVVTQSPRNKVAVTGEKVTLSCNQTNNHNNMYWYRQDTGHELRLIHYSLGIRNVISGDIPDGYKASRPSQENFSLILELATPSQTSVYFCASGGGGTLYFGAGTRLSVL

SEQ ID NO: 54 - protein, 110 aa, Mus musculus; identical to SEQ ID NO: 27

SEQ ID NO: 55 - protein, 110 aa, Mus musculus
EAVVTQSPRNKVAVTGEKVTLSCNQTNNHNNMYWYRQDTGHELRLIHYSLGIRNVRPGDIPDGYKASRPSQENFSLILELATPSQTSVYFCASGGGGTLYFGAGTRLSVL

SEQ ID NO: 56 - protein, 110 aa, Mus musculus
EAVVTQSPRNKVAVTGEKVTLSCNQTNNHNNMYWYRQDTGHELRLIHYSLGIHNVEKGDIPDGYKASRPSQENFSLILELATPSQTSVYFCASGGGGTLYFGAGTRLSVL

SEQ ID NO: 57 - protein, 110 aa, Mus musculus; identical to SEQ ID NO: 28

SEQ ID NO: 58 - protein, 110 aa, Mus musculus
EAVVTQSPRNKVAVTGEKVTLSCNQTNNHNNMYWYRQDTGHELRLIFGSYGIHNVEKGDIPDGYKASRPSQENFSLILELATPSQTSVYFCASGGGGTLYFGAGTRLSVL

SEQ ID NO: 59 - protein, 110 aa, Mus musculus
EAVVTQSPRNKVAVTGEKVTLSCNQTNNHNNMYWYRQDTGHELRLIFRSLGIRNVEKGDIPDGYKASRPSQENFSLILELATPSQTSVYFCASGGGGTLYFGAGTRLSVL

SEQ ID NO: 60 - protein, 110 aa, Mus musculus
EAVVTQSPRNKVAVTGEKVTLSCNQTNNHNNMYWYRQDTGHELRLIFASLGIHNVEKGDIPDGYKASRPSQENFSLILELATPSQTSVYFCASGGGGTLYFGAGTRLSVL

SEQ ID NO: 61 - protein, 110 aa, Mus musculus
EAVVTQSPRNKVAVTGEKVTLSCNQTNNHNNMYWYRQDTGHELRLIFRSLGIHNVEKGDIPDGYKASRPSQENFSLILELATPSQTSVYFCASGGGGTLYFGAGTRLSVL

SEQ ID NO: 62 - protein, 110 aa, Mus musculus; identical to SEQ ID NO: 29

SEQ ID NO: 63 - protein, 110 aa, Mus musculus
EAVVTQSPRNKVAVTGEKVTLSCSQTNNHNNMYWYRQDTGHELRLIFMSHGIRNVEKGDIPDGYKASRPSQENFSLILELATPSQTSVYFCASGGGGTLYFGAGTRLSVL

SEQ ID NO: 64 - protein, 110 aa, Mus musculus
EAVVTQSPRNKVAVTGEKVTLSCSQTNNHNNMYWYRQDTGHELRLIFASLGIHNVEKGDIPDGYKASRPSQENFSLILELATPSQTSVYFCASGGGGTLYFGAGTRLSVL

SEQ ID NO: 65 - protein, 110 aa, Mus musculus
EAVVTQSPRNKVAVTGEKVTLSCSQTNNHNNMYWYRQDTGHELRLIFRSLGIRNVEKGDIPDGYKASRPSQENFSLILELATPSQTSVYFCASGGGGTLYFGAGTRLSVL

SEQ ID NO: 66 - protein, 111 aa, Mus musculus; identical to SEQ ID NO: 32

SEQ ID NO: 67 - protein, 111 aa, Mus musculus; identical to SEQ ID NO: 33

SEQ ID NO: 68 - protein, 111 aa, Mus musculus; identical to SEQ ID NO: 34

SEQ ID NO: 69 - protein, 111 aa, Mus musculus; identical to SEQ ID NO: 36

SEQ ID NO: 70 - protein, 111 aa, Mus musculus; identical to SEQ ID NO: 35

SEQ ID NO: 71 - protein, 110 aa, Mus musculus; identical to SEQ ID NO: 38

SEQ ID NO: 72 - protein, 111 aa, Mus musculus; identical to SEQ ID NO: 30

SEQ ID NO: 73 - protein, 111 aa, Mus musculus
EAVVTQSPRNKVAVTGEKVTLSCKQTCSYLDNMYWYRQDTGHELRLIFMSHGIRNVEKGDIPDGYKASRPSQENFSLILELATPSQTSVYLCASGGGGTLYFGAGTRLSVL

SEQ ID NO: 74 - protein, 118 aa, Homo sapiens; identical to SEQ ID NO: 20

SEQ ID NO: 75 - protein, 118 aa, Homo sapiens
GAVVSQHPSMVIVKSGTSVKIECRSLDTNIHTMFWYRQFPKQSLMLMATSHEGFNAIYEQGVTKDKFLINHASPTLSTLTVTSAHPEDSGFYVCSALAGSGSSTDTQYFGPGTQLTVL

SEQ ID NO: 76 - protein, 111 aa, Mus musculus; identical to SEQ ID NO: 36

SEQ ID NO: 77 - protein, 111 aa, Mus musculus
EAVVTQSPRNKVAVTGEKVTLSCKQTNSYFDNMYWYRQDTGHELRLIHYSYGVGNTEKGDIPDGYEASRLTWRTFSLILVSATPSQTSVYFCASGGGGTLYFGAGTRLSVL

SEQ ID NO: 78 - protein, 118 aa, Homo sapiens; identical to SEQ ID NO: 1

SEQ ID NO: 79 - protein, 110 aa, Mus musculus; identical to SEQ ID NO: 24